(19) United States
(12) Patent Application Publication
(10) Pub. No.: US 2023/0181686 A1
(43) Pub. Date: Jun. 15, 2023

(54) COMPOSITIONS AND METHODS FOR TREATING NEOVASCULARIZATION AND ISCHEMIC RETINOPATHIES BY TARGETING ANGIOGENESIS AND CHOLESTEROL TRANSPORT

(71) Applicants: Baylor College of Medicine, Houston, TX (US); The Methodist Hospital d/b/a Houston Methodist, Houston, TX (US)

(72) Inventors: Yingbin Fu, Houston, TX (US); Longhou Fang, Houston, TX (US); Mackenzie Parker, Houston, TX (US); Megan Shen, Houston, TX (US); Qing Yan, Houston, TX (US); Nduka Enemchukwu, Houston, TX (US)

(21) Appl. No.: 17/999,348

(22) PCT Filed: Jun. 21, 2021
(86) PCT No.: PCT/IB2021/055463

§ 371 Date: Nov. 18, 2022

Related U.S. Application Data
(60) Provisional application No. 63/030,421, filed on May 27, 2020.

(51) Int. Cl. A61K 38/17; A61P 27/02

(52) U.S. Cl. CPC A61K 38/1709; A61P 27/02

(57) ABSTRACT

Embodiments of the disclosure include methods and compositions for the treatment of neovascularization- and ischemic retinopathy-related disorders. In some embodiments, a composition comprising an effective amount of an apoA-I binding protein or its agonist in combination with anti-VEGF reagents is administered to an individual in need thereof to treat, prevent, reverse, and/or meliorate conditions associated with macular degeneration or cancer. In some embodiments, a composition comprising an effective mount of an AIBP-inhibitor is administered to an individual in need thereof to stimulate revascularization in the eye to treat, prevent, reverse, and/or ameliorate conditions associated with ischemic retinopathies.

This application claims priority to U.S. Provisional Patent Application Ser. No. 63/030,421, filed May 27, 2020, which is incorporated by reference herein in its entirety.

### TECHNICAL FIELD

Embodiments of the disclosure include at least the fields of cell biology, molecular biology, and medicine.

### BACKGROUND

Age-related macular degeneration (AMD) is a major cause of blindness in older people. The wet form (choroidal neovascularization, CNV) underlies ˜90% of blindness cases in AMD. In addition to wet AMD, CNV occurs in other diseases including ocular histoplasmosis, angioid streaks, pathological myopia, and choroidal ruptures. CNV can be treated by regular injections of anti-VEGF agents (ranibizumab, bevacizumab, and aflibercept, etc.). However, a significant number of patients have a poor response or are nonresponsive to standardized treatment with anti-VEGF agents, and in some cases, patients experience a slow loss of efficacy of anti-VEGF agents after repeated administration over time. Thus, new treatment methods that can overcome resistance to anti-VEGF agents would be useful in treating CNV.

Aberrant angiogenesis, or neovascularization, is also a hallmark of cancer and is essential for tumor growth and metastasis. Although anti-VEGF therapy has provided clinical benefit for cancer patients, resistance to these agents often emerges, and tumor growth resumes, along with rapid revascularization following termination of these therapies. Thus, new treatment methods that can overcome the anti-VEGF resistance would be useful in treating cancer characterized by neovascularization.

Ischemic retinopathies, such as retinopathy of prematurity (ROP) and diabetic retinopathy (DR), are also characterized by neovascularization and are the main causes of severe visual impairment in children and adults (with diabetes), respectively. Ischemic retinopathies are characterized by an initial phase of loss of the preexisting retinal blood vessels and sustained ischemia that leads to a secondary abnormal vessel growth phase, or neovascularization phase, into the vitreous humor, which can result in retinal detachment and blindness. The standard-of-care laser photocoagulation for ROP is invasive and may permanently reduce the visual field in addition to inducing myopia. Since restoration of local vascular supply via reparative angiogenesis is crucial for the preservation of neural function in ischemic tissues (Chopp et al., 2007; Joyal et al., 2011; Li et al., 2007; Wei et al., 2015), new treatment strategies to promote physiological revascularization are highly desirable for ischemic retinopathies.

The present disclosure provides a solution to the need for treatment of certain medical conditions characterized by neovascularization, or abnormal growth of new blood vessels, including at least AMD, cancer, and ischemic retinopathies.

### BRIEF SUMMARY

Neovascularization is the natural formation of new blood vessels, usually in the form of functional microvascular networks, capable of perfusion by red blood cells. Inhibiting vascular endothelial growth factor (VEGF), a signal protein produced by cells that stimulates the formation of blood vessels, can treat or prevent neovascularization. However, decreased efficacy of and resistance to anti-VEGF agents prevents effective treatment of medical conditions characterized by neovascularization. Thus, embodiments of the disclosure concern methods and compositions for more effective prevention and/or treatment of medical conditions characterized by neovascularization, including at least wet AMD, cancer, and ischemic retinopathy. Particular embodiments of the disclosure utilize at least apoA-I binding protein or an inhibitor thereof for treating medical conditions characterized by neovascularization or any medical condition in which the blocking of new blood vessel formation is beneficial. In specific embodiments, the medical condition is treated, or at least one symptom is improved upon, following blockage of new blood vessel formation.

In a particular embodiment, the disclosure concerns methods of treating or preventing neovascularization in an individual, comprising providing to the individual an effective amount of one or more compositions that comprise two or more compounds whose combination treats or prevents neovascularization in the individual. In specific embodiments, one of the compounds facilitates therapeutic efficacy of the other compound. In specific cases, one of the compounds overcomes resistance in an individual with respect to the other compound. In certain cases, multiple compounds are provided to an individual regardless of whether or not resistance to one of the compounds has been demonstrated in the individual. In some cases, multiple compounds are provided to the individual when the individual has one or more risk factors for developing resistance to one of the compounds. In particular embodiments, use of multiple compounds in the individual has an additive or synergistic therapeutic effect in the individual. In particular embodiments, one of the compounds is apoA-I binding protein (AIBP) or a functionally active fragment or derivative thereof, and one of the compounds is an anti-VEGF agent, and one of the compounds is apoA-I.

In one embodiment, provided is a method of treating or preventing neovascularization in an individual, comprising the step of delivering to the individual a therapeutically effective amount of a composition comprising apoA-I binding protein (AIBP) or a functionally active fragment or derivative thereof. The neovascularization can be associated with age-related macular degeneration and/or cancer. The neovascularization can be associated with resistance to anti-VEGF agents and/or aberrant new blood vessel formation.

In some embodiments, the fragment comprises the N-terminus, the C-terminus, both the N-terminus and C-terminus, or neither of the N-terminus or C-terminus. In some embodiments, the fragment or derivative is at least 70, 75, 80, 85, 90, 91, 92, 93, 94, 95, 96, 97, 98, or 99% identical to SEQ ID NO:1. In some embodiments, the derivative comprises 1, 2, 3, 4, 5, or more variations compared to SEQ ID NO:1.

Treatment with the AIBP composition can improve removal of cholesterol from macrophages, reduces inflammation, and restores macrophages' ability to inhibit angiogenesis, thereby treating or preventing neovascularization. Provision of the AIBP composition can reduce resistance to anti-VEGF agents. Therefore, embodiments of the disclosure include treatment with AIBP composition, one or more anti-VEGF agents, and optionally apoA-I.

Any composition can be delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage. The AIBP composition may be delivered to the individual multiple times. The AIBP composition may be delivered to the individual once a day, more than once a day, more than once a week, more than once a month, or more than once a year. The AIBP composition may be provided to the individual by constant infusion. The AIBP composition may be delivered in the same or different formulations as one or more anti-VEGF agents and/or apoA-1.

In some embodiments, the individual is provided one or more additional therapies for treating or preventing neovascularization. The second therapy comprises one or more anti-VEGF agents. The anti-VEGF agent can comprise one or more antibodies selected from the group consisting of brolucizumab, pegaptanib, bevacizumab, ranibizumab, and afilbercept. The anti-VEGF agent comprises one or more small molecules selected from the group consisting of lapatinib, sunitinib, sorafenib, axitinib, pazopanib, and AZ2171 (cediranib). The anti-VEGF agent may be provided before, during, or after provision of the AIBP composition and/or before, during, or after apoA-1.

In one embodiment, provided is a method of treating or preventing pathological neovascularization in an individual, comprising the step of delivering to the individual a therapeutically effective amount of an anti-apoA-I binding protein (AIBP) agent. The neovascularization can be associated with ischemic retinopathy, which can be retinopathy of prematurity (ROP), diabetic retinopathy (DR), or central retinal vein occlusion. The neovascularization can be associated with aberrant new blood vessel formation in the vitreous humor.

Treatment with the anti-AIBP agent can inhibit AIBP, and inhibition of AIBP can promote VEGFR2 signaling and inhibit Notch1 signaling. Treatment with the anti-AIBP agent can also inhibit pathological neovascularization and/or promote regenerative revascularization.

The anti-AIBP agent can be delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage. The anti-AIBP agent can be delivered to the individual multiple times. The anti-AIBP agent can be delivered to the individual once a day, more than once a day, more than once a week, more than once a month, or more than once a year. The anti-AIBP agent can be provided to the individual by constant infusion. The anti-AIBP agent can comprise anti-AIBP antibodies, antisense nucleotides, and/or small molecule antagonists of AIBP.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1 shows that AIBP suppresses choroid sprouting. n=3 per group. p=0.035

FIG. 2 shows that AIBP inhibited old macrophage's capacity to promote HRMEC angiogenesis. Young, young macrophages; old, old macrophages. **, p<0.01.

FIG. 3 illustrates a process for producing oxygen-induced retinopathy (OIR) in mice. Neonatal mice are exposed to 75% oxygen from P7 to P12 to induce vessel loss and returned to room air from P12 to P17 to induce pathologic neovascularization. The peak of neovascularization tufts occurs at P17.

FIG. 4 shows that no significant difference was observed in the avascular area between WT and Apoa1bp^{-/-} retinas at P12. n=5 for Apoa1bp^{-/-}. n=4 for WT.

FIGS. 5A-5D show that genetic ablation of Apoa1bp accelerates vascular regrowth and decreases pathological neovascularization. (FIG. 5A & FIG. 5C), Retinal whole-mount of WT (Apoa1bp^{+/+}) and Apoa1bp^{-/-} mice at P17 of OIR stained with Alexa 568-isolectin. Avascular area was outlined by a gold line, and neovascular areas were highlighted in white. Quantification of avascular (FIG. 5B) and neovascular areas (FIG. 5D). Data represent mean±SEM; n=5. **p<0.01, ***p<0.001.

FIGS. 6A-6C show that AIBP overexpression impeded reparative angiogenesis and reduced pathological angiogenesis. (FIG. 6A), Diagram illustrating the strategy to generate the Apoa1bp^{OE} knock-in mice, which enable global Dox-induced expression of human AIBP. SS: MMP9 secretion signal. (FIG. 6B) The lactating females were fed Dox-containing food or control food after delivering the P0 neonatal mice, and AIBP expression was assessed. Quantification of avascular (FIG. 6C) and neovascular areas (FIG. 6D) of Apoa1bp^{OE} mice at P17 of OIR. Data represent mean±SEM; n=5-10. **p<0.01, ***p<0.001.

FIGS. 7A and 7B show that AIBP neutralization by pAb and mAb abolished the inhibitory effect of AIBP on HRMEC angiogenesis. HRMECs were treated with AIBP and HDL3 combination, or AIBP and HDL3 preincubated with AIBP antibodies as indicated. The tube length was quantified from three repeats. Scale bar: 100 μm. **, p<0.01. pAb, polyclonal antibody; mAb, mouse monoclonal antibody.

FIG. 8 shows that 1-month Apoa1bp mice showed normal retinal structure. Bright field retinal section was overlaid with DAPI nuclear staining. ROS, rod outer segment; RIS, rod inner segment; ONL, outer nuclear layer; INL, inner nuclear layer; IPL, inner plexiform layer; GCL, ganglion cell layer. Scale, 20 μm.

FIGS. 9A-9D show scotopic and photopic ERGs of Apoa1bp^{-/-} and WT control mice. (FIGS. 9A & 9B) Scotopic a- and b-wave amplitudes, respectively, as a function of light intensity. (FIGS. 9C & 9D) Photopic green and UV b-wave amplitudes, respectively, as a function of light intensity. Mean±SEM. n=3 (WT); 7 (Apoa1bp^{-/-}).

FIGS. 10A-10E show AIBP enhances cholesterol efflux and inhibits angiogenesis on retinal and choroidal ECs. (FIG. 10A) Effect of AIBP and HDL3 on lipid rafts of HRMECs. Cells were stained for lipid rafts (green, D4-EGFP) and nuclei (blue, DAPI) after preincubation with control media, AIBP, HDL3, or AIBP plus HDL3. (FIG. 10B) Quantification of the area of lipid rafts per cell. n=92, 53, 70, and 86 cells for control, AIBP, HDL3, and AIBP+HDL3, respectively. (FIG. 10C) Effect of AIBP and HDL3 on EC tube formation using the Matrigel-based assay. The length of HRMEC tubes was quantified after various treatments. n=12, 9, 6, 6, and 6 cells for control, AIBP, HDL3, AIBP+HDL3, and MβCD, respectively. Representative images (FIG. 10D) and quantification of microvascular sprouting area (FIG. 10E) after AIBP treatment. n=3 explants per group. Data represent mean±SEM in FIGS. 10B, 10C, 10E. *p<0.05, **p<0.01, ***p<0.001; by one-way analysis of variance (ANOVA) with Tukey post hoc analysis (FIGS. 10B, 10C) two-tailed Student's t-test=(FIG. 10E).

FIGS. 11A-11D show AIBP treatment reduces intracellular lipid accumulation in macrophages isolated from 8- and 18-month old mice and suppresses old macrophages' ability to promote HRMEC angiogenesis. (FIG. 11A), Peritoneal macrophage isolated from 1-, 8-, and 18-month 57B16/J mice were treated with AIBP and apoA-I, and stained with oil red O. (FIG. 11B), Quantification on the number of lipid droplets per cell in macrophages. n=25 (control) and 28 (AIBP/apoA-I) macrophages derived from 8-month mice, 43 (control) and 39 (AIBP/apoA-I) macrophages derived from 18-month mice. (FIG. 11C), Effect of AIBP on old macrophages' ability to promote angiogenesis of HRMECs. Macrophages (MΦ) isolated from 1-month and 18-month mice were pretreated with different combination of AIBP and apoA-I and co-cultured with HRMECs. Scale bar, 1000 μm. (FIG. 11D), Quantitative analysis of total segment length of HRMECs in c. n=7 (without co-culture), 10 (1-month MΦ+control), 6 (1-month MΦ+AIBP/apoA-I), 14 (18-month MΦ+control), and 13 (18-month MΦ+AIBP/apoA-I) HRMECs. Data represent mean±SEM in B, D. **p<0.01, ***p<0.001; by two-tailed Student's t-test (FIG. 11B) or one-way ANOVA with Tukey post hoc analysis (FIG. 11D).

FIGS. 12A-12C show AIBP deficiency profoundly increases choroid sprouting and laser-induced CNV. (FIG. 12A), Representative images (left panels) and quantification of microvascular sprouting area (right panel) from Apoa1bp^{-/-} and WT adult mouse choroid explants. n=3 choroid explants for WT, 4 for Apoa1bp^{-/-}. (FIG. 12B), Representative images of CNV lesions labeled by Alexa 568-isolectin on RPE choroid flatmounts (left panels) and quantification of CNV areas (right panel) from Apoa1bp^{-/-} and WT adult mice. n=33 laser spots per group. (FIG. 12C), Representative images of CNV lesions (left panels) and quantification of CNV areas (right panel) after intravitreal injection of a rabbit anti-AIBP antibody or a control IgG. n=28 laser spots per group. Scale bar is 20 μm in (FIG. 12B) and (FIG. 12C). Data represent mean±SEM in (FIGS. 12A-C). *p<0.05, ***p<0.001 by two-tailed Student's t-test.

FIGS. 13A-13B show AIBP expression in mouse CNV, non-lesion, and control (non-laser) retinal areas. (FIG. 13A), AIBP mRNA (in red) detected by RNAscope counter stained by hematoxylin II. The magnified images on the top show AIBP in RPE (orange arrowheads) and CNV membranes (orange arrows). * indicates CNV membranes in the left panel. Scale, 20 μm in magnified images and 40 μm in others. (FIG. 13B), Quantification of AIBP mRNA in the Cho-RPE, photoreceptors, and inner retina. n=4 retinas for CNV (including CNV and non-lesion areas) and non-laser control. Data represent mean±SEM. NS, p>0.05, **p<0.01, ***p<0.001 by one-way ANOVA with Tukey post hoc analysis. Cho, choroid.

FIGS. 14A-141 show AIBP expression in human CNV, non-lesion, and normal control retinas detected by RNAscope. (FIGS. 14B, 14D, 14E) Representative RNAscope images of non-lesion, CNV, and normal retinas, respectively, show AIBP expression and localization. (FIGS. 14A & 14C) H&E staining of adjacent sections for FIGS. 14B & 14D, respectively. (FIGS. 14F, 14G, 14H) Magnified image of dashed orange box in FIGS. 14B, 14D, and 14E, respectively. (FIG. 14I) Quantification of AIBP mRNA. n=5, 4, and 4 retinas for CNV, non-lesion areas, and normal, respectively. Data represent mean±SEM. NS, p>0.05, *p<0.05, **p<0.01 by one-way ANOVA with Tukey post hoc analysis. Yellow arrows, orange arrowheads, and orange arrows indicate AIBP expression in the inner segment, RPE, and choroid, respectively, in the normal and non-lesion areas. Black arrowheads and the black arrow indicate AIBP expression in the RPE and choroid, respectively, in CNV. * indicates subretinal disciform scar in CNV. Scale bar, 20 μm in FIGS. 14A-E14, 10 μm in FIGS. 14F-14H.

FIGS. 15A-15F show a comparison between AIBP/apoA-I, anti-VEGF agent, and combination treatment in suppressing laser-induced CNV in mice. (FIG. 15A), Representative images of CNV lesions after different combinations of AIBP and apoA-I treatment in 8-10 week-old mice. (FIG. 15B), Quantification of CNV areas in (FIG. 15A). n=23 (BSA control), 19 (apoA-I), 23 (AIBP), and 20 (AIBP+apoA-I) laser spots. (FIGS. C & D), Comparison of AIBP/apoA-I and an anti-VEGF antibody in inhibiting laser-induced CNV in 6-8 weeks (FIG. 15C) and 8-month (FIG. 15D) mice. n=14 (control), 18 (AIBP+apoA-I), and 17 (anti-VEGF) laser spots in (C), and 37 (control), 47 (AIBP+apoA-I), and 43 (anti-VEGF) laser spots in (FIG. 15D). (FIG. 15E), Quantification on the effect of anti-VEGF (low, 5 ng), anti-VEGF (high, 25 ng), AIBP+apoA-I, and AIBP+apoA-I+anti-VEGF (low) in suppressing laser-induced CNV in 18-month male mice. n=20 (control), 15 (anti-VEGF low), 26 (anti-VEGF high), 19 (AIBP+apoA-I), and 20 (AIBP+apoA-I+anti-VEGF low) laser spots. (FIG. 15F), Macrophage depletion by C12MDP in old mice restored CNV sensitivity to anti-VEGF treatment and blunted the synergistic effect of combination therapy. n=23 (control), 24 (C12MDP), 16 (C12MDP+anti-VEGF low), 24 (C12MDP+AIBP+apoA-I+anti-VEGF low) laser spots. Data represent mean±SEM. NS, p>0.05, *p<0.05, **p<0.01 by one-way ANOVA with Tukey post hoc analysis.

FIGS. 16A-16B show AIBP suppresses old macrophage's ability to promote angiogenesis of HRMECs. Representative images (FIG. 16A) and quantitative analysis (FIG. 16B) of total segment length of HRMECs co-cultured with peritoneal macrophages isolated from 1-month and 8-month mice. Macrophages were pretreated with different combinations of AIBP and apoA-I. n=7 (1-month MΦ+control), 6 (1-month MΦ+AIBP/apoA-I), 9 (8-month MΦ+control), and 6 (8-month MΦ+AIBP/apoA-I) HRMECs. Data represent mean±SEM. *P<0.05, **p<0.01 by one-way ANOVA with Tukey post hoc analysis. Scale bar, 1000 μm in (A).

FIGS. 17A-17B show AIBP neutralization abolishes the inhibitory effect of AIBP on HRMEC angiogenesis. (FIG. 17A) Effect of AIBP pAb antibody neutralization on the inhibitory effect of AIBP on HRMEC tube formation. Scale bar, 1000 μm. (FIG. 17B) Quantification of the total tube length. n=4 per group. Data represent mean±SEM. ***p<0.001 by one-way ANOVA with Tukey post hoc analysis. pAb, polyclonal antibody.

FIG. 18 shows AIBP expression in the retina of WT but not Apoa1bp^{-/-} (KO) mice. AIBP mRNA was detected by RNAscope (in red). White arrows indicate AIBP expression in the RPE. Nuclei were labeled with DAPI (blue). Fluorescent images were overlaid with bright field to show RPE and choroid. Scale bar, 10 μm.

FIG. 19 shows a negative control probe targeting bacterial dihydrodipicolinate reductase shows no signal on human retinal section. Human retinal paraffin sections were probed with either the negative control probe against bacterial dihydrodipicolinate reductase or the AIBP probe. Scale bar, 20 μm.

FIGS. 20A-20B shows a dose-range study for AIBP and anti-VEGF in inhibiting laser-induced CNV. (FIG. 20A), Dose-ranging study for AIBP and apoA-I. n=21-30 per dose. (FIG. 20B), Dose-ranging study for an anti-VEGF neutralizing antibody. n=25-49 per dose. Data represent mean±SEM.

FIG. 21 shows a comparison between AIBP/apoA-I, anti-VEGF agent, and combination treatment in suppressing laser-induced CNV in old female mice. Quantification on the effect of anti-VEGF (5 ng), AIBP+apoA-I, and AIBP+apoA-I+anti-VEGF in suppressing laser-induced CNV in 14-15 months female mice. n=30 (BSA control), 26 (anti-VEGF), 23 (AIBP+apoA-I), and 22 (AIBP+apoA-I+anti-VEGF) laser spots. Data represent mean±SEM. *p<0.05 by one-way ANOVA with Tukey post hoc analysis.

FIGS. 22A-22B. Vascular morphology in laser-induced CNV assessed by Alexa 568-isolectin-labeled flatmount. (22A) Young vs. old mice. (22B) Control vs. AIBP/apoA-I/anti-VEGF treated old mice. White arrowheads and arrows indicate branching arterioles and vascular loops in old mice, respectively. Scale, 20 μm.

### DETAILED DESCRIPTION

### I. Exemplary Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. The terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. The term “about” is used according to its plain and ordinary meaning in the area of cell and molecular biology to indicate that a value includes the standard deviation of error for the device or method being employed to determine the value. The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Recitation of ranges of values herein are merely intended to serve as a shorthand method of referring individually to each separate value falling within the range, unless otherwise indicated herein, and each separate value is incorporated into the specification as if it were individually recited herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment.

Furthermore, the particular features, structures, or characteristics may be combined in any suitable manner in one or more embodiments. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The terms “subject,” “individual,” and “patient” and “individual” may be used interchangeably and typically comprise a mammal, in certain embodiments a human or a non-human primate. While the compositions and methods are described herein with respect to use in humans, they are also suitable for animal, e.g., veterinary use. Thus certain illustrative organisms include, but are not limited to humans, non-human primates, canines, equines, felines, porcines, ungulates, lagomorphs, and the like. Accordingly, certain embodiments contemplate the compositions and methods described herein for use with domesticated mammals (e.g., canine, feline, equine), laboratory mammals (e.g., mouse, rat, rabbit, hamster, guinea pig), and agricultural mammals (e.g., equine, bovine, porcine, ovine), and the like. The term “subject” does not require one to have any particular status with respect to a hospital, clinic, or research facility (e.g., as an admitted patient, a study participant, or the like). Accordingly, in various embodiments, the subject can be a human (e.g., adult male, adult female, adolescent male, adolescent female, male child, female child) under the care of a physician or other health worker in a hospital, psychiatric care facility, as an outpatient, or other, clinical context. In certain embodiments, the subject may not be under the care or prescription of a physician, or other, health worker. In certain embodiments the subject may not be under the care a physician or health worker and, in certain embodiments, may self-prescribe and/or self-administer the compounds described herein. The subject may be of any gender, race, or age.

As used herein, the phrase “subject in need thereof” or “individual in need thereof” refers to a subject or individual, as described infra, that suffers or is at a risk of suffering (e.g., pre-disposed such as genetically pre-disposed, or subjected to environmental conditions that pre-dispose, etc.) from the diseases or conditions listed herein (e.g., neovascularization).

As used herein, the term “therapeutically effective amount” is synonymous with “effective amount”, “therapeutically effective dose”, and/or “effective dose” refers to an amount of an agent sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., preventing aberrant new blood vessel growth. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be partially reduced or completed eliminated, and so forth.

As used herein, the terms “treatment,” “treat,” or “treating” refers to intervention in an attempt to alter the natural course of the individual or cell being treated, and may be performed either for prophylaxis or during the course of pathology of a disease or condition such as for example neovascularization. Treatment may serve to accomplish one or more of various desired outcomes, including, for example, preventing occurrence or recurrence of disease, alleviation of symptoms, and diminishment of any direct or indirect pathological consequences of the disease, lowering the rate of disease progression, amelioration or palliation of the disease state, remission or improved prognosis, and/or producing some desired effect, e.g., preventing aberrant new blood vessel growth. The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel.

### II. ApoA-I Binding Protein (AIBP)

In particular embodiments, the disclosure concerns methods and compositions comprising AIBP to treat or prevent medical conditions associated with neovascularization. Neovascularization can be associated with resistance to anti-VEGF agents and can be characterized by aberrant new blood vessel formation. AIBP is effective in removing cholesterol from macrophages, reducing inflammation, inhibiting macrophages' ability to promote angiogenesis, disrupting VEGFR2 signaling, and promoting Notch1 signaling. Particular embodiments of the disclosure utilize apoA-I binding protein for treating medical conditions characterized by neovascularization or any medical condition in which the blocking of new blood vessel formation is beneficial. In specific embodiments, the medical condition is treated, or at least one symptom is improved upon, following blockage of new blood vessel formation.

AIBP is a secreted protein discovered in a screen of proteins that physically associate with ApoA-I. It is also known as apolipoprotein A-I binding protein, AI-BP, apoA-I binding protein, NAD(P)H-hydrate epimerase, NAXE, NAD(P)HX epimerase, PEBEL, YJEFN1, and APOA1BP. ApoA-I has a specific role in lipid metabolism and is the major protein component of HDL particles in plasma. The ApoA-I protein, as a component of HDL particles, enables efflux of fat molecules by accepting fats from within cells (including macrophages within the walls of arteries which have become overloaded with ingested fats from oxidized LDL particles) for transport (in the water outside cells) elsewhere, including back to LDL particles or to the liver for excretion. Cholesterol is a structural component of the cell, indispensable for normal cellular function, but its excess often leads to abnormal proliferation, migration, inflammatory responses and/or cell death. To prevent cholesterol overload, ATP-binding cassette (ABC) transporters mediate cholesterol efflux from the cells to apolipoprotein A-I (ApoA-I) and to the ApoA-I-containing high-density lipoprotein (HDL). Maintaining efficient cholesterol efflux is essential for normal cellular function. Human APOA1BP mRNA encoding the AIBP protein is ubiquitously expressed. AIBP binding to ApoA-I implies that AIBP may modulate HDL function and play a role in cholesterol efflux.

ApoA-I binding protein (AIBP) has been shown to accelerate cholesterol efflux from endothelial cells (EC) to HDL and thereby regulate angiogenesis. AIBP/HDL-mediated cholesterol depletion reduces lipid rafts, interferes with VEGFR2 dimerization and signaling, and inhibits VEGF-induced angiogenesis in vitro and mouse aortic neovascularization ex vivo. Remarkably, AIBP has also been shown to regulate the membrane lipid order in embryonic zebrafish vasculature and functions as a non-cell autonomous regulator of zebrafish angiogenesis. AIBP knockdown results in dysregulated sprouting/branching angiogenesis, while forced AIBP expression inhibits angiogenesis. Dysregulated angiogenesis is phenocopied in Abca1/Abcg1-deficient embryos, and cholesterol levels are increased in AIBP-deficient and Abca1/Abcg1-deficient embryos. These data demonstrate that secreted AIBP positively regulates cholesterol efflux from EC and that effective cholesterol efflux is critical for proper angiogenesis.

Further, when fed a high-cholesterol, high-fat diet, Apoa1bp^{-/-} Ldlr^{-/-} mice, which do not express AIBP or low density lipoprotein receptor, have been shown to exhibit exacerbated weight gain, liver steatosis, glucose intolerance, hypercholesterolemia, hypertriglyceridemia, and larger atherosclerotic lesions compared with Ldlr^{-/-} mice. Feeding Apoa1bp^{-/-} Ldlr^{-/-} mice a high-cholesterol, normal-fat diet did not result in significant differences in lipid levels or size of atherosclerotic lesions from Ldlr^{-/-} mice. Conversely, adeno-associated virus-mediated overexpression of AIBP reduced hyperlipidemia and atherosclerosis in high-cholesterol, high-fat diet-fed Ldlr^{-/-} mice. Injections of recombinant AIBP reduced aortic inflammation in Ldlr^{-/-} mice fed a short high-cholesterol, high-fat diet. Conditional overexpression of AIBP in zebrafish also reduced diet-induced vascular lipid accumulation. In experiments with isolated macrophages, AIBP facilitated cholesterol efflux to HDL, reduced lipid rafts content, and inhibited inflammatory responses to lipopolysaccharide. These data demonstrate that AIBP confers protection against diet-induced metabolic abnormalities and atherosclerosis.

In another study, ApoE−/− mice with established atherosclerotic plaques were infected with rAAV-AIBP or rAAV-AIBP(Δ115-123), respectively. AIBP-treated mice showed a reduction of atherosclerotic lesion formation, an increase in circulating HDL levels, and an enhancement of reverse cholesterol transport to the plasma, liver, and feces. AIBP increased ABCA1 protein levels in the aorta and peritoneal macrophages. Furthermore, AIBP diminished atherosclerotic plaque macrophage content and the expression of chemotaxis-related factors. In addition, AIBP prevented macrophage inflammation by inactivating NF-κB and promoted the expression of M2 markers like Mrc-1 and Arg-1. However, lack of 115-123 amino acids of AIBP(Δ115-123) had no such preventive effects on the progression of atherosclerosis. These data demonstrate that AIBP inhibits atherosclerosis progression.

In summary, AIBP can enhance cholesterol efflux in endothelial cells (ECs) and macrophages and suppress angiogenesis by modulating both VEGFR2 and Notch1 signaling, two important pathways in both physiological and pathological angiogenesis (13,23). In ECs, AIBP binds apoA-I containing high density lipoprotein (HDL) and accelerates cholesterol efflux, which reduces lipid raft content and inhibits lipid raft-anchored VEGFR2 signaling, to thereby limit angiogenesis (13). In macrophages, AIBP binds to toll-like receptor 4 (TLR4) in cholesterol-laden or inflamed macrophages/microglia to augment cholesterol efflux, normalize plasma lipid rafts, and decrease inflammation (14,15).

### III. Anti-AIBP and Anti-VEGF Agents

In some embodiments, an effective amount of an AIBP composition is administered to treat, prevent, reverse, and/or ameliorate medical conditions associated with neovascularization. In some embodiments, the AIBP composition is administered alone or in combination with one or more additional therapies for treating or preventing neovascularization, which can be an AIBP agonist and/or anti-VEGF agent. In other embodiments, an effective amount of an AIBP inhibitor or Anti-AIBP agent is administered to treat, prevent, reverse, and/or ameliorate medical conditions associated with neovascularization.

Anti-AIBP agents can include but are not limited to lipids, carbohydrates, small molecules, antibodies, nucleic acids, or mimetic polypeptides. For example, anti-AIBP agents can comprise anti-AIBP antibodies, antisense nucleotides, blocking peptides, and/or small molecule antagonists. As used herein, an “AIBP inhibitor” or “anti-AIBP agents” or grammatical variations thereof refers to an agent that inhibits the expression or activity of an AIBP protein or polypeptide, including variants or isoforms thereof. The inhibition may be to an extent (in magnitude and/or spatially), and/or for a time, sufficient to produce the desired effect. Inhibition may be prevention, retardation, reduction or otherwise hindrance of AIBP expression and/or activity. Such inhibition may be in magnitude and/or be temporal or spatial in nature. Inhibition of expression of AIBP can be assessed using methods well known in the art to measure transcription and/or protein production. The expression and/or activity of AIBP can be inhibited by an agent by at least or about 10%, 15%, 20%, 25%, 30%, 35%, 40%, 45%, 50%, 55%, 60%, 65%, 70%, 75%, 80%, 85%, 90%, 95% or more compared to the expression and/or activity of AIBP in the absence of the inhibitor. An AIBP inhibitor may be specific or selective for AIBP or may be capable of inhibiting the expression or activity of one or more other proteins or polypeptides in addition to AIBP. Furthermore, an AIBP inhibitor may act directly or indirectly on AIBP. Accordingly the inhibitor may operate directly or indirectly on AIBP proteins or polypeptides, an AIBP mRNA or gene, or alternatively act via the direct or indirect inhibition of any one or more components of an AIBP-associated pathway. Such components may be molecules activated, inhibited or otherwise modulated prior to, in conjunction with, or as a consequence of AIBP polypeptide or protein activity.

As used herein, “AIBP activity” or an “activity of AIBP” refers to any activity associated with AIBP polypeptides and/or AIBP proteins, including, but not limited to, the ability of AIBP to remove cholesterol from endothelial cells and/or macrophages, reduce inflammation, restore macrophages' ability to inhibit angiogenesis which leads to neovascularization, disrupt VEGFR2 signaling, and/or promote Notch1 signaling.

The term “inhibiting” and variations thereof such as “inhibition” and “inhibits” as used herein in relation to activity of AIBP means complete or partial inhibition of characteristics, including, but not limited to, the ability of AIBP to remove cholesterol from macrophages, reduce inflammation, restore macrophages' ability to inhibit angiogenesis which leads to neovascularization, disrupt VEGFR2 signaling, and/or promote Notch1 signaling. The inhibition may be to an extent (in magnitude and/or spatially), and/or for a time, sufficient to produce the desired effect. Inhibition may be prevention, retardation, reduction or otherwise hindrance of activity or activation of AIBP. Such inhibition may be in magnitude and/or be temporal or spatial in nature. Removal of cholesterol from macrophages, reduction in inflammation, restoration of macrophages' ability to inhibit angiogenesis which leads to neovascularization, disruption of VEGFR2 signaling, and/or promotion of Notch1 signaling by an agent (i.e. an AIBP inhibitor or antagonist) can be assessed by measuring removal of cholesterol from macrophages, reduction in inflammation, restoration of macrophages' ability to inhibit angiogenesis which leads to neovascularization, disruption of VEGFR2 signaling, and/or promotion of Notch1 signaling in the presence and absence of the agent following an event that would normally trigger removal of cholesterol from macrophages, reduction in inflammation, restoration of macrophages' ability to inhibit angiogenesis which leads to neovascularization, disruption of VEGFR2 signaling, and/or promotion of Notch1 signaling. The removal of cholesterol from macrophages, reduction in inflammation, restoration of macrophages' ability to inhibit angiogenesis which leads to neovascularization, disruption of VEGFR2 signaling, and/or promotion of Notch1 signaling can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to the removal of cholesterol from macrophages, reduction in inflammation, restoration of macrophages' ability to inhibit angiogenesis which leads to neovascularization, disruption of VEGFR2 signaling, and/or promotion of Notch1 signaling in the absence of exposure to the agent. Inhibition of the AIBP activity by an agent (i.e. an AIBP inhibitor) can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to AIBP activity in the absence of exposure to the agent.

As used herein the term “expression” may refer to expression of a polypeptide or protein, or to expression of a polynucleotide or gene, depending on the context. Expression of a polynucleotide may be determined, for example, by measuring the production of RNA transcript levels. Expression of a protein or polypeptide may be determined, for example, by immunoassay using an antibody(ies) that bind with the polypeptide.

Anti-VEGF agents include antibodies against VEGF. Antibodies against VEGF were first developed as an intravenous treatment for metastatic colorectal cancer. The anti-VEGF agents for intravitreal use are brolucizumab, pegaptanib (Macugen™, Eyetech/Pfizer), bevacizumab (Avastin™), conbercept (Lumitin™), and ranibizumab (Avastin® and Lucentis™, both from Genentech/Roche). Pegaptanib sodium is a 50-kDa aptamer; a pegylated modified oligonucleotide that adopts a specific 3D configuration and has a high affinity for extracellular VEGF-165. Studies have proven its effect on inhibiting pathological neovascularization and vascular leakage in rodents with induced macular choroidal neovascularization. Ranibizumab is a shorter 48-kDa antibody fragment (κ isotype) that binds to the receptors of biologically active VEGF-A, including VEGF-110. This blocks the binding of VEGF-A to VEGR receptor (VEGFR)1 and VEGFR2 receptors on endothelial cells. Bevacizumab, however, is a larger whole antibody of 149 kDa, and possesses two antigen-binding domains for its receptors Flt-1 and KDR. It binds to all isoforms of VEGF. The difference in molecular masses may determine their potential difference in efficacy and their duration of action. As described in Jeganathan et al., Curr. Opin. Ophthalmol., 2009, and Tolentino, Surv. Ophthalmol., 2011, detailed safety profiles and risks of adverse effects are now available for these agents as they have been used extensively in patients for the treatment of age-related macular degeneration. Furthermore, the incidence of raised IOP and development of lens opacity with anti-VEGF agents is negligible when compared with intravitreal steroid injections. Afilbercept, a recombinant fusion protein consisting of vascular endothelial growth factor (VEGF)-binding portions from the extracellular domains of human VEGF receptors 1 and 2, that are fused to the Fc portion of the human IgG1 immunoglobulin, has also been shown to effectively inhibit VEGF.

Anti-VEGF agents can also include orally-available small molecules that inhibit the tyrosine kinases stimulated by VEGF: lapatinib, sunitinib, sorafenib, axitinib, and pazopanib. AZ2171 (cediranib), a multi-targeted tyrosine kinase inhibitor, has also been shown to have anti-edema effects by reducing the permeability and aiding in vascular normalization. Recombinant vectors expressing VEGF-neutralizing proteins including AAV2-sFLT-1 and/or AAV2-sFLT01 have also been implicated as anti-VEGF agents. Finally, afilbercept, a recombinant fusion protein consisting of vascular endothelial growth factor (VEGF)-binding portions from the extracellular domains of human VEGF receptors 1 and 2, that are fused to the Fc portion of the human IgG1 immunoglobulin, has also been shown to effectively inhibit VEGF.

### IV. ApoA-1

In some embodiments, an effective amount of ApoA-1 is administered to treat, prevent, reverse, and/or ameliorate medical conditions associated with neovascularization. In some embodiments, ApoA-1 is administered alone or in combination with one or more additional therapies for treating or preventing neovascularization, which can be an AIBP agonist and/or anti-VEGF agent. In other embodiments, an effective amount of an AIBP inhibitor or Anti-AIBP agent is administered to treat, prevent, reverse, and/or ameliorate medical conditions associated with neovascularization.

An example of an ApoA-1 protein is available at GenBank® Accession No. NP_000030, which is incorporated by reference herein in its entirety. When utilized with AIBP and/or one or more anti-VEGF agents, the ApoA-1 protein may or may not be in the same formulation. In some cases, a functional fragment of the ApoA-1 protein is used, such as 10, 20, 30, 40, 50, 100, 150, 200, or 250 or more contiguous amino acids of the protein.

```
(SEQ ID NO: 3)
    1 mkaavltlav lfltgsqarh fwqqdeppgs
        pwdrvkdlat vyvdvikdsg rdyvsqfegs
  61 algkqlnlkl idnwdsvtst fsklreqlgp
        vtqefwdnle keteglrqem skdleevkak
121 vqpylddfqk kwqeemelyr qkveplrael
        qegarqklhe iqeklsplge emrdrarahv
181 dalrthlapy sdelrqrlaa rlealkengg
        arlaeyhaka tehlstlsek akpaledlrq
241 gllpvlesfk vsflsaleey tkklntq
```

### V. Age-Related Macular Degeneration

In particular embodiments, the disclosure concerns methods and compositions comprising a combination of AIBP and anti-VEGF reagents and optionally ApoA-1 to overcome resistance to anti-VEGF agents and increase therapeutic efficacy for wet AMD. In specific cases, delivery of AIBP to an individual removes cholesterol from macrophages, reduces inflammation, inhibits macrophages' ability to promote angiogenesis which leads to neovascularization, disrupts VEGFR2 signaling, and/or promotes Notch1 signaling.

Age-related macular degeneration (AMD) is the leading cause of irreversible blindness in the elderly. In the U.S., the number of AMD cases is expected to double from 11 million to nearly 22 million by 2050. In the world, the projected number of people with AMD is 196 million in 2020 and 288 million in 2040, and the estimated global cost of AMD is $343 billion (1).

AMD can be classified into wet (choroidal neovascularization, CNV) and dry (geographic atrophy) forms. CNV underlies ˜90% of cases of blindness due to AMD. In addition to wet AMD, CNV occurs in other diseases including ocular histoplasmosis, angioid streaks, pathological myopia, and choroidal ruptures. CNV can be treated by regular injections of anti-VEGF reagents (ranibizumab, bevacizumab, and aflibercept, etc.). Although anti-VEGF agents have improved the treatment for wet AMD and CNV, a significant number of patients are unresponsive to standardized treatment with anti-VEGF agents or experience a slow loss of efficacy of anti-VEGF agents after repeated administration over time (3). For example, up to one-fourth of all treated patients, defined as non-responders, do not benefit from anti-VEGF therapy, with visual acuity deteriorating over time despite treatment. Anti-VEGF treatment efficacy declines in 51% of patients receiving intravitreal ranibizumab, and 67% of patients treated with bevacizumab have persistent subretinal fluid (Comparison of Age-related Macular Degeneration Treatments Trials (CATT) Research Group et al., 2016). 19.7%-36.6% of patients have persistent active exudation even after 1 year of regular aflibercept treatments (Heier et al., 2012). The mean visual acuity also gradually decreased during long-term follow-up with a pro re nata retreatment when patients exited from the MARINA or ANCHOR trial (SEVEN-UP Study) (6).

Thus, resistance (or diminished response) to anti-VEGF therapy represents an unmet clinical need for the treatment of wet AMD. Efforts to develop new treatments are hampered by poor understanding of the mechanisms underlying anti-VEGF resistance and the lack of suitable AMD animal models that exhibit anti-VEGF resistance.

Previous studies have shown that macrophages have increased density and proliferative activity in response to bevacizumab treatment in surgically excised human CNV membranes (7), suggesting that macrophages play a role in anti-VEGF resistance. Reduced cholesterol efflux in old macrophages promotes CNV formation (8), and macrophage depletion inhibits experimental CNV (9,10), further implicating macrophages as a key player in CNV pathogenesis. Finally, VEGF165 acts as a proinflammatory cytokine, targeting monocytes, macrophages, and leukocytes in a positive feedback loop that involves endothelial cells (ECs) to sustain pathological neovascularization process (11,12).

### VI. Neovascularization in Cancer

In particular embodiments, the disclosure concerns methods and compositions comprising a combination of AIBP and anti-VEGF reagents to overcome resistance to anti-VEGF agents and increase therapeutic efficacy for neovascularized cancers. In specific cases, delivery of AIBP to an individual removes cholesterol from macrophages, reduces inflammation, restores macrophages' ability to inhibit angiogenesis which leads to neovascularization, disrupts VEGFR2 signaling, and/or promotes Notch1 signaling. In specific cases, the cancer is a solid tumor cancer.

Angiogenesis is a hallmark of cancer and is essential for tumor growth and metastasis (Hanahan and Weinberg, 2000). The VEGF pathway is the dominant pathway regulating blood vessel formation in cancer. However, clinical survival benefits from anti-VEGF therapies such as bevacizumab have been modest. Resistance to these agents often emerges, and tumor growth resumes, along with rapid revascularization, following these therapies (Burger et al., 2011; Hayes, 2011; Mancuso et al., 2006). New treatment methods that can overcome the anti-VEGF resistance are highly significant in improving the efficacy for cancer treatment.

Studies have shown that macrophages in the tumor microenvironment orchestrate anti-VEGF therapy resistance (Dalton et al., 2017). Further, tumor angiogenesis shares similarities with ocular angiogenesis such as CNV. In fact, the anti-VEGF reagent bevacizumab was originally developed to target tumor angiogenesis, and it was later discovered that bevacizumab is quite effective in treating CNV. Another anti-VEGF reagent, aflibercept, was also approved by FDA to treat both CNV and cancer.

Cancers that can be treated or prevented with the methods and compositions of the disclosure include but are not limited to: Acute Lymphoblastic Leukemia (ALL); Acute Myeloid Leukemia (AML); Adrenocortical Carcinoma; AIDS-Related Cancers, including Kaposi Sarcoma (Soft Tissue Sarcoma), AIDS-Related Lymphoma (Lymphoma), and Primary CNS Lymphoma (Lymphoma); Anal Cancer; Astrocytomas (Brain Cancer); Atypical Teratoid/Rhabdoid Tumor, Childhood, Central Nervous System (Brain Cancer); Basal Cell Carcinoma of the Skin; Bile Duct Cancer; Bladder Cancer; Bone Cancer (includes Ewing Sarcoma and Osteosarcoma and Malignant Fibrous Histiocytoma); Brain Tumors; Breast Cancer; Bronchial Tumors (Lung Cancer); Burkitt Lymphoma; Carcinoid Tumor (Gastrointestinal); Carcinoma of Unknown Primary; Cardiac (Heart) Tumors; Central Nervous System Cancers, including Atypical Teratoid/Rhabdoid Tumor (Brain Cancer), Medulloblastoma and Other CNS Embryonal Tumors (Brain Cancer), Germ Cell Tumor (Brain Cancer), and Primary CNS Lymphoma; Cervical Cancer; Childhood Cancers; Cancers of Childhood, Unusual; Cholangiocarcinoma; Chordoma (Bone Cancer); Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell Lymphoma (Mycosis Fungoides and Sézary Syndrome); Ductal Carcinoma In Situ (DCIS); Embryonal Tumors, Medulloblastoma and Other Central Nervous System (Brain Cancer); Endometrial Cancer (Uterine Cancer); Ependymoma (Brain Cancer); Esophageal Cancer; Esthesioneuroblastoma (Head and Neck Cancer); Ewing Sarcoma (Bone Cancer); Extracranial Germ Cell Tumor; Extragonadal Germ Cell Tumor; Eye Cancer, including Intraocular Melanoma and Retinoblastoma; Fallopian Tube Cancer; Fibrous Histiocytoma of Bone, Malignant, and Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors, including Childhood Central Nervous System Germ Cell Tumors (Brain Cancer), Childhood Extracranial Germ Cell Tumors, Extragonadal Germ Cell Tumors, Ovarian Germ Cell Tumors, and Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer (Head and Neck Cancer); Intraocular Melanoma; Islet Cell Tumors, Pancreatic Neuroendocrine Tumors; Kaposi Sarcoma (Soft Tissue Sarcoma); Kidney (Renal Cell) Cancer; Langerhans Cell Histiocytosis; Laryngeal Cancer (Head and Neck Cancer); Leukemia; Lip and Oral Cavity Cancer (Head and Neck Cancer); Liver Cancer; Lung Cancer (Non-Small Cell, Small Cell, Pleuropulmonary Blastoma, and Tracheobronchial Tumor); Lymphoma; Male Breast Cancer; Malignant Fibrous Histiocytoma of Bone and Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary (Head and Neck Cancer); Midline Tract Carcinoma With NUT Gene Changes; Mouth Cancer (Head and Neck Cancer); Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer (Head and Neck Cancer); Nasopharyngeal Cancer (Head and Neck Cancer); Neuroblastoma; Non-Hodgkin Lymphoma; Non-Small Cell Lung Cancer; Oral Cancer, Lip and Oral Cavity Cancer and Oropharyngeal Cancer (Head and Neck Cancer); Osteosarcoma and Malignant Fibrous Histiocytoma of Bone; Ovarian Cancer; Pancreatic Cancer; Pancreatic Neuroendocrine Tumors (Islet Cell Tumors); Papillomatosis (Childhood Laryngeal); Paraganglioma; Paranasal Sinus and Nasal Cavity Cancer (Head and Neck Cancer); Parathyroid Cancer; Penile Cancer; Pharyngeal Cancer (Head and Neck Cancer); Pheochromocytoma; Pituitary Tumor; Plasma Cell Neoplasm/Multiple Myeloma; Pleuropulmonary Blastoma (Lung Cancer); Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood (Soft Tissue Sarcoma); Salivary Gland Cancer (Head and Neck Cancer); Sarcoma, including Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, and Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic (Head and Neck Cancer); Stomach (Gastric) Cancer; T-Cell Lymphoma, Cutaneous; Testicular Cancer; Throat Cancer (Head and Neck Cancer), including Nasopharyngeal Cancer, Oropharyngeal Cancer, and Hypopharyngeal Cancer; Thymoma and Thymic Carcinoma; Thyroid Cancer; Tracheobronchial Tumors (Lung Cancer); Transitional Cell Cancer of the Renal Pelvis and Ureter (Kidney (Renal Cell) Cancer); Unknown Primary, Carcinoma of; Unusual Cancers of Childhood; Ureter and Renal Pelvis, Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; and Wilms Tumor and Other Childhood Kidney Tumors.

### VII. Ischemic Retinopathy

In particular embodiments, the disclosure concerns methods and compositions comprising an inhibitor of AIBP to treat or prevent ischemic retinopathy and/or retinopathy related disorders. Ischemic retinopathies, such as retinopathy of prematurity (ROP), diabetic retinopathy (DR), and central retinal vein occlusion, are the main causes of severe visual impairment in children and adults with diabetes (Hartnett, 2017; Kempen et al., 2004).

Ischemic retinopathies are characterized by an initial phase of loss of the preexisting vessel bed and sustained hypoxia that leads to a secondary vasoproliferative phase characterized by vessel proliferation into the vitreous humor, which can result in retinal detachment and blindness. Current treatments only target pathological neovascularization (NV) rather than addressing ischemia, which is the root cause of the NV, and have many limitations. For example, the standard-of-care laser photocoagulation for ROP is invasive and may permanently reduce the visual field in addition to inducing myopia (Ospina et al., 2005). Intravitreal anti-VEGF treatment has been associated with reactivation of ROP (Hu et al., 2012; Snyder et al., 2016) and suppression of systemic VEGF that may affect body growth and organ development in preterm infants (Haigh, 2008; Wu et al., 2015). Even in adult in DR, chronic anti-VEGF therapy may increase photoreceptor and retinal pigment epithelial atrophy as well as subretinal fibrosis since VEGF is essential for the maintenance of the choriocapillaris and photoreceptors (Kurihara et al., 2012; Saint-Geniez et al., 2009).

Physiological revascularization is highly desirable in ischemic retinopathies to restore metabolic supply, improve retinal function, and reduce the retinal ischemia that drives the detrimental pathologic neovascularization. Since restoration of local vascular supply via reparative angiogenesis is crucial for the preservation of neural function in ischemic tissues (Chopp et al., 2007; Joyal et al., 2011; Li et al., 2007; Wei et al., 2015), new treatment strategies to promote physiological revascularization are highly desirable for ischemic retinopathies.

A critical element of ischemic retinopathies is the inadequate revascularization of the ischemic retina that leads to intravitreal neovascularization. Further, AIBP is expressed in retinal neurons, and recent studies suggest that neurovascular crosstalk (i.e. interaction between retinal neurons and retinal blood vessels) plays a critical role in shaping vascular regeneration in the ischemic retina (Fukushima et al., 2011; Joyal et al., 2011; Sapieha, 2012; Wei et al., 2015).

### VIII. AIBP Compositions

In embodiments of the disclosure, there are compositions that encompass part or all of AIBP. Although the AIBP may be from any mammal, including mice, rat, chimpanzee, dog, cat, cow, pig, and so forth, but in specific embodiments the AIBP is from human. This is because the AIBP sequence homology between different animals is high. Although the AIBP composition may be isolated from a mammal (and may or may not be modulated thereafter), in specific embodiments the AIBP composition is synthetically generated, such as by recombinant means. The AIBP may be synthetically produced, in certain cases. The AIBP may be recombinant human AIBP or naturally occurring AIBP. In particular cases, the AIBP is administered exogenously (and therefore external to cells). In specific embodiments, the AIBP is recombinant. Other forms of AIBP are also contemplated for use in embodiments of the present disclosure, including but not limited to AIBP which has been modified post-translationally in vivo or in vitro, including but not limited to the following post-translational modifications: hydroxylation, methylation, ubiquitylation, sulfation, phosphorylation, glycosylation, lipidation, carbonylation, carbamylation, acylation, alkylation, biotinylation, oxidation, amidation, isoprenylation, prenylation, glipyatyon, lipoylation, phosphopantetheinylation, pegylation, racemization, amide bond formation, protein splicing, formation of disulfide bonds, addition of smaller chemical groups, addition of cofactors for enhanced activity, addition of hydrophobic groups for membrane localization, cleavage of peptide bonds, or a combination thereof.

In particular embodiments, the AIBP composition comprises the entirety of SEQ ID NO:1, although in other embodiments the AIBP composition may comprise a functionally equivalent derivative of SEQ ID NO: 1. The term “functionally equivalent derivative” refers to a polynucleotide or polypeptide sequence that has been modified by substitution, insertion or deletion of one or more (e.g., 1, 2, 3, 4, 5, 6, 7, 8, 9 10) nucleotides or one or more (e.g., 1, 2, 3, 4, 5, 6, 7, 8, 9, 10) amino acids, respectively, but that has substantially the same or better activity as the reference sequence. Function of a polypeptide may be assessed experimentally, for example, by determining activity in an in vitro or in vivo experiment. Whether or not a given polypeptide or polynucleotide is “functional” may be determined by first selecting an appropriate function to assess. For example, functionality of AIBP variants may be assessed in vitro or in vivo for the ability of the variants to attenuate neovascularization, for example, by improving removal of cholesterol from endothelial cells and/or macrophages, reducing inflammation, and/or restoring macrophages' ability to inhibit angiogenesis. Functionality of AIBP variants may also be assessed in vitro or in vivo for the ability of the variants to increase the efficacy of an anti-VEGF agent in inhibiting angiogenesis. In some cases, a functional molecule may be one that exhibits the desired function to a statistically significant degree (e.g. p<0.05; <0.01; <0.001). As a reference sequence, a AIBP polypeptide sequence is in the National Center for Biotechnology Information's GenBank® database at Accession Number AJ315849.

```
AJ315849
(SEQ ID NO: 1)
    1 MSRLRALLGL GLLVAGSRVP RIKSQTIACR SGPTWWGPQR
        LNSGGRWDSE
  51 VMASTVVKYL SQEEAQAVDQ ELFNEYQFSV DQLMELAGLS
        CATAIAKAYP
101 PTSMSRSPPT VLVICGPGNN GGDGLVCARH LKLFGYEPTI
        YYPKRPNKPL
151 FTALVTQCQK MDIPFLGEMP AEPMTIDELY ELVVDAIFGF
        SFKGDVREPF
201 HSILSVLKGL TVPIASIDIP SGWDVEKGNA GGIQPDLLIS
        LTAPKKSATQ
251 FTGRYHYLGG RFVPPALEKK YQLNLPPYPD TECVYRLQ
```

In specific embodiments, an AIBP composition comprises a fragment of AIBP that is at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or 460 amino acids in length. In some embodiments, an AIBP composition comprises a fragment of AIBP that is no more than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or 460 amino acids in length. As an alternative to, or in addition to, the fragment having a certain length, the fragment may comprise sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1.

In one embodiment, there is provided an isolated human AIBP polypeptide fragment comprising at least a functional portion of AIBP (SEQ ID NO:1), or a functionally equivalent fragment or derivative thereof. In some embodiments, the polypeptide comprises at least 10, 11, 12, 13, 14, 15, 16, 17, 18, 19, 20, 21, 22, 23, 24, 25, 26, 27, 28, 29, 30, 31, 37, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300 or more contiguous amino acids of AIBP (SEQ ID NO: 1). In some embodiments, the functional derivative comprises 1, 2, 3, 4, or 5 amino acid differences, such as conservative amino acid modifications, compared to SEQ ID NO:1. The AIBP fragment may include an N-terminal and/or C-terminal truncation of SEQ ID NO:1, such as of 1, 2, 3, 4, 5, 6, 7, 8, 9, 10, 15, 20, 25, or more amino acids truncated from the N-terminal and/or C-terminal of SEQ ID NO:1.

In some embodiments, any AIBP polypeptide fragment is less than 300, 275, 250, 200, 100, 90, 80, 70, 60, 50, 40, 30, 20, or 10 amino acids in length. In some embodiments any AIBP polypeptide fragment is between 1-300, 50-300, 100-300, or 200-300 amino acids in length. In some embodiments the polypeptide fragment is between 10-60, 20-60, 30-60, 40-60 or 50-60 amino acids in length.

The corresponding polynucleotide that encodes SEQ ID NO:1 is SEQ ID NO:2:

```
      1 gccgggggcg cgcgctctgc gagctggatg
          tccaggctgc gggcgctgct gggcctcggg
    61 ctgctggttg cgggctcgcg cctgccgcgg
          atcaaaagcc agaccatcgc ctgtcgctcg
  121 ggacccacct ggtggggacc gcagcggctg
          aactcgggtg gccgctggga ctcagaggtc
  181 atggcgagca cggtggtgaa gtacctgagc
          caggaggagg cccaggccgt ggaccaggag
  241 ctatttaacg aataccagtt cagcgtggac
          caacttatgg aactggccgg gctgagctgt
  301 gctacagcca tcgccaaggc atatcccccc
          acgtccatgt ccaggagccc ccctactgtc
  361 ctggtcatct gtggcccggg gaataatgga
          ggagatggtc tggtctgtgc tcgacacctc
  421 aaactctttg gctacgagcc aaccatctat
          taccccaaaa ggcctaacaa gcccctcttc
  481 actgcattgg tgacccagtg tcagaaaatg
          gacatccctt tccttgggga aatgcccgca
  541 gagcccatga cgattgatga actgtatgag
          ctggtggtgg atgccatctt tggcttcagc
  601 ttcaagggcg atgttcggga accgttccac
          agcatcctga gtgtcctgaa gggactcact
  661 gtgcccattg ccagcatcga cattccctca
          ggatgggacg tggagaaggg aaatgctgga
  721 gggatccagc cagacttgct catctccctc
          acagccccca aaaaatctgc aacccagttt
  781 accggtcgct accattacct ggggggtcgt
          tttgtgccac ctgctctgga aaagaagtac
  841 cagctgaacc tgccacccta ccctgacact
          gagtgtgtct atcgtctgca gtgagggaag
  901 gtgggtgggt attctcccca ataaagactt
          agagcccctc tcttccagaa ctgtggattc
  961 ctgggagctc ctctggcaat aaaagtcagt
          gaatggtgga agtcagagag caaccctggg
1021 gattgggtgc catctctcta ggggtaacac
          aaagggcaag aggttgctat ggtatttgga
1081 acacatgaaa atggactgtt agatgccaaa
          aaaaaaaaaa
```

In particular, embodiments, the AIBP composition is formulated as a pharmaceutical composition. Pharmaceutical compositions of the present disclosure comprise an effective amount of one or more AIBP compositions dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to an animal, such as, for example, a human, as appropriate, and do not interfere with the therapeutic methods of the disclosure. The preparation of a pharmaceutical composition that contains at least one AIBP composition or additional active ingredient will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The AIBP compositions may comprise different types of carriers depending on whether it is to be administered in solid, liquid or aerosol form, and whether it needs to be sterile for such routes of administration, such as injection. The AIBP compositions of the present disclosure can be administered intravenously, intradermally, transdermally, intrathecally, intraarterially, intraperitoneally, intranasally, intravaginally, intrarectally, topically, intramuscularly, subcutaneously, mucosally, intratumorally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The AIBP composition(s) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in practicing the methods of the present disclosure is appropriate. Examples of carriers or diluents include fats, oils, water, saline solutions, lipids, liposomes, resins, binders, fillers, alcohols, and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art. The AIBP composition may be lyophilized.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may include the use of a pharmaceutical lipid vehicle compositions that incorporates a AIBP composition, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the AIBP composition(s) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to the subject can be determined by physical and physiological factors such as body weight, severity of condition, the type of disease being treated, previous or concurrent therapeutic interventions, idiopathy of the subject and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% (by weight) of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration of the active agent, e.g., an AIBP composition according to the present disclosure, and any range derivable therein. In non-limiting examples of a derivable range from the numbers listed herein, a range of about 1 mg/kg body weight to about 100 mg/kg body weight, about 5 microgram/kg body weight to about 500 milligram/kg body weight, etc., of the active agent can be administered, based on the numbers described above.

Alimentary Compositions and Formulations

In particular embodiments of the present disclosure, the AIBP composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the AIBP compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10% (by weight), and preferably about 1% to about 2% (by weight).

Parenteral Compositions and Formulations

In further embodiments, the AIBP compositions may be administered via a parenteral route. As used herein, the term “parenteral” includes routes that bypass the alimentary tract. Specifically, the pharmaceutical compositions disclosed herein may be administered for example, but not limited to intravitreally, intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Compositions and Formulations

In other preferred embodiments of the disclosure, the AIBP composition may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical AIBP compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Examples of Methods of Treatment or Prevention of AMD and Cancer

Embodiments of the disclosure include methods of delivering a therapeutically effective amount of one or more AIBP compositions to an individual in need thereof. In particular embodiments, a therapeutically effective amount of one or more AIBP inhibitors is administered to an individual in need thereof. In specific embodiments, the individual has or is at risk for having neovascularization. The individual may have a condition that has as a symptom and/or a mechanism an aberrant increase in angiogenesis, for example. Embodiments of the disclosure include treatment or prevention of any medical condition in which modulation of neovascularization would be beneficial. In specific embodiments, an individual is provided a therapeutically effective amount of one or more AIBP compositions or AIBP inhibitors for attenuation of neovascularization in an individual, including when the individual has dysregulation of physiological processes following AMD, neovascularized cancers, and/or ischemic retinopathies which can lead to neovascularization.

In specific embodiments, the medical condition treated or prevented with AIBP or an AIBP inhibitor comprises AMD or neovascularized cancers, and/or ischemic retinopathies which can lead to neovascularization. In particular embodiments, neovascularization is not treated with compositions and methods of the disclosure. In some cases, the AIBP or AIBP inhibitor treats or prevents the medical condition in the individual by inhibiting neovascularization, for example.

Embodiments of the disclosure include compositions and methods that prevent the development of neovascularization or the progression to neovascularization as a result of AMD, neovascularized cancers, and/or ischemic retinopathies. In specific cases, delivery of AIBP to an individual removes cholesterol from macrophages, reduces inflammation, inhibits macrophages' ability to promote angiogenesis which leads to neovascularization, disrupts VEGFR2 signaling, and/or promotes Notch1 signaling. In some cases, once an individual appears to be at risk for developing neovascularization, an individual is given an effective amount of one or more AIBP compositions or AIBP inhibitors as part of their care.

Although in some cases the AIBP composition or AIBP inhibitor is provided as a sole therapy for the individual, in some cases the individual is provided a one or more additional therapies for treating or preventing neovascularization. The one or more additional therapies may be of any kind, but in specific cases the one or more additional therapies is an AIBP agonist, an anti-VEGF agent, or a combination thereof. AIBP compositions or AIBP inhibitors may also be an additional therapy to attenuate neovascularization until the primary process is resolved (e.g., resolution of AMD, cancer, or ischemic retinopathy).

In particular embodiments, an individual that is at risk for neovascularization as a result of AMD, cancer, or ischemic retinopathy or that is known to have AMD, cancer, or ischemic retinopathy is provided a therapeutically effective amount of one or more AIBP compositions or one or more AIBP inhibitors. In some cases, the individual has been diagnosed with AMD, cancer, or ischemic retinopathy, for example. In some cases, the individual is at risk of developing AMD, cancer, or ischemic retinopathy. Risk factors for developing AMD include but are not limited to advanced age, smoking, family history of disease, gender, race, prolonged sun exposure, diet, obesity, high blood pressure, eye color, inactivity, and presence of AMD in one eye. Risk factors for developing cancer include but are not limited to advanced age, family history of disease, smoking, obesity, alcohol use, certain types of viral infections, certain chemicals, and exposure to radiation. Risk factors for developing ischemic retinopathy, specifically diabetic retinopathy, include but are not limited to diabetes, duration of diabetes, poor control of blood sugar, high blood pressure, high cholesterol, pregnancy, smoking, and race. Risk factors for developing ischemic retinopathy, specifically retinopathy of prematurity, include but are not limited gestational age, low birth weight, hypoxia, duration of oxygen supplementation, respiratory distress syndrome, twin pregnancy, anemia, blood transfusions, sepsis, intraventricular hemorrhage, hypotension, and hypothermia. An individual characterized by one or more of these risk factors may be provided an effective amount of one or more AIBP compositions or AIBP inhibitors.

In some embodiments, a medical condition is treated or prevented with an AIBP composition or AIBP inhibitor that is delivered to the individual multiple times, such as once a day, more than once a day, one a week, more than once a week, once a month, more than once a month, once a year, or more than once a year. The multiple treatments may or may not have the same formulations and/or routes of administration(s). Any administration may be as a continuous infusion.

The provider skilled in the art of medical care and decision may determine an appropriate end-point for AIBP composition or AIBP inhibitor therapy based on the specific disease process and clinical course of the patient or individual.

### V. Kits

Any of the compositions described herein may be part of a kit. The kits may comprise a suitably aliquoted AIBP composition or AIBP inhibitor of the present disclosure, and the component(s) of the kits may be packaged either in aqueous media or in lyophilized form. The container of the kits will generally include at least one vial, test tube, flask, bottle, syringe or other container, into which a component may be placed, and preferably, suitably aliquoted. Where there is more than one component in the kit, the kit also will generally contain a second, third or other additional container into which the additional component(s) may be separately placed. However, various combinations of components may be comprised in a vial. The kits of the present disclosure also will typically include a container for holding the AIBP composition or AIBP inhibitor and any other reagent containers in close confinement for commercial sale.

When the components of the kit are provided in one and/or more liquid solutions, the liquid solution is an aqueous solution, with a sterile aqueous solution being contemplated. The compositions may also be formulated into a syringeable composition. In which case, the container may itself be a syringe, pipette, and/or other such like apparatus, from which the formulation may be applied to a particular area of the body, injected into an individual, and/or even applied to and/or mixed with the other components of the kit. However, the component(s) of the kit may be provided as dried powder(s). When reagents and/or components are provided as a dry powder, the powder can be reconstituted by the addition of a suitable solvent. It is envisioned that the solvent may also be provided in another container.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### AIBP Potently Inhibits Choroid Angiogenesis Ex Vivo

AIBP is a secreted protein-apoA-I binding protein that effectively inhibits angiogenesis by accelerating cholesterol efflux from endothelial cells (ECs) to high-density lipoprotein (HDL), disrupting cholesterol-rich lipid rafts and impairing VEGFR2 signaling, thereby limiting angiogenesis (13). AIBP shows a remarkable capability to restrict angiogenesis in vitro and in vivo (13). Conversely, AIBP ablation increases lipid raft abundance, bolsters VEGFR2 signaling and angiogenesis in zebrafish and mice (13; 23). The inventors have shown that AIBP limits angiogenesis in human umbilical vein endothelial cells (HUVECs), aortic neovascularization, and retinal angiogenesis in mice (13; 23).

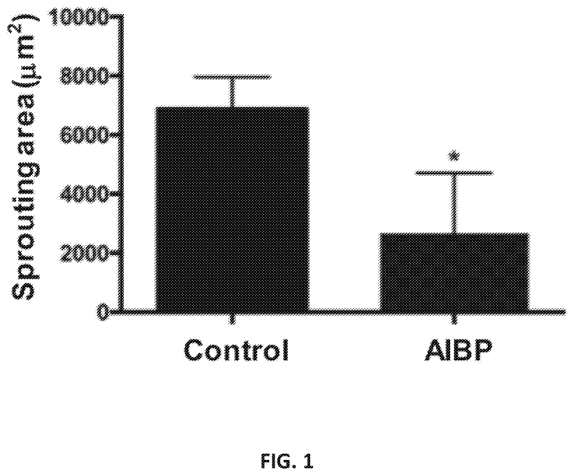

To explore the role of AIBP in choroid angiogenesis, the inventors used the ex vivo model of choroid sprouting, which has been shown to be a reproducible model of choroid angiogenesis (Shao et al., 2013). Recombinant AIBP potently inhibited choroid sprouting (61% reduction, p=0.035, FIG. 1), consistent with the inhibitory role of AIBP in angiogenesis. Note, choroid explants were cultured 4 days in complete EGM-2 media, which contains HDL from bovine serum, and therefore no need to add exogenous apoA-I or HDL.

### Example 2

### AIBP Enhances Cholesterol Efflux from Old Macrophages and Inhibits their Capacity to Promote Angiogenesis

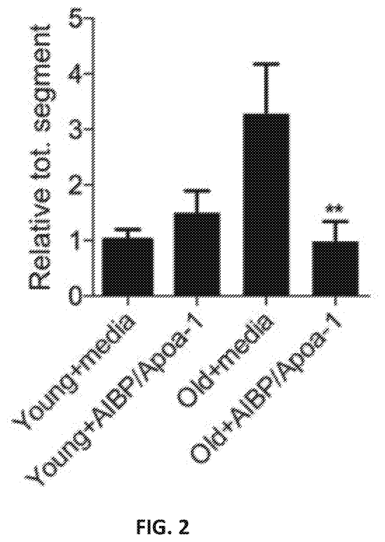

Previous studies have shown that old macrophages accumulated more cholesterol and promote angiogenesis compared with young ones (Sene et al., 2013). Indeed, the inventors found that co-culture of primary human retinal microvascular ECs (HRMECs) with peritoneal old macrophages from 18-month C57BL/6J mice had 3.2 times more proliferation compared with HRMECs co-cultured with peritoneal young macrophages from 6-8-week mice (FIG. 2). Treatment with AIBP and apoA-I reduced cholesterol accumulation in old macrophages and inhibited their ability to promote HRMEC proliferation (FIG. 2). Since impaired cholesterol efflux in old macrophages promotes CNV formation (Sene et al., 2013), this result suggests that AIBP can inhibit CNV by enhancing cholesterol removal from old macrophages. The ability of AIBP to inhibit neovascularization suggests it can target tumor angiogenesis.

### Example 3

### AIBP Deficiency Promoted Reparative Angiogenesis and Reduced

### Pre-Retinal Neovascularization in OIR

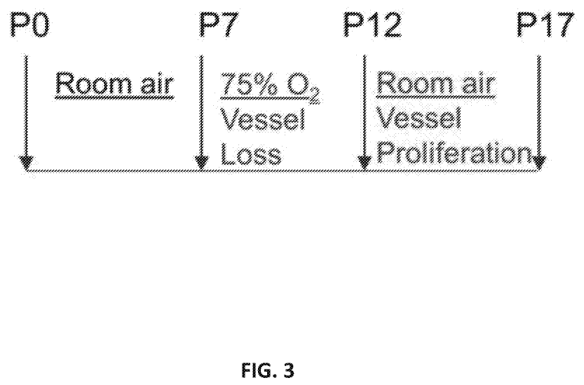

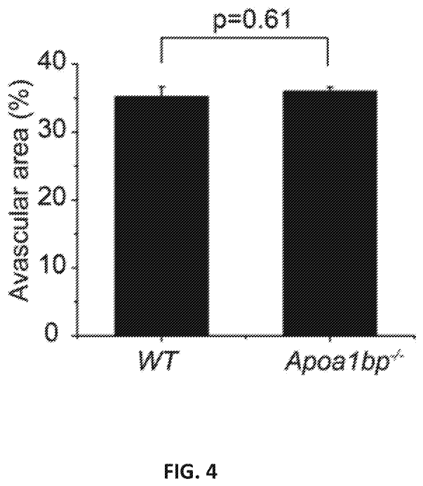

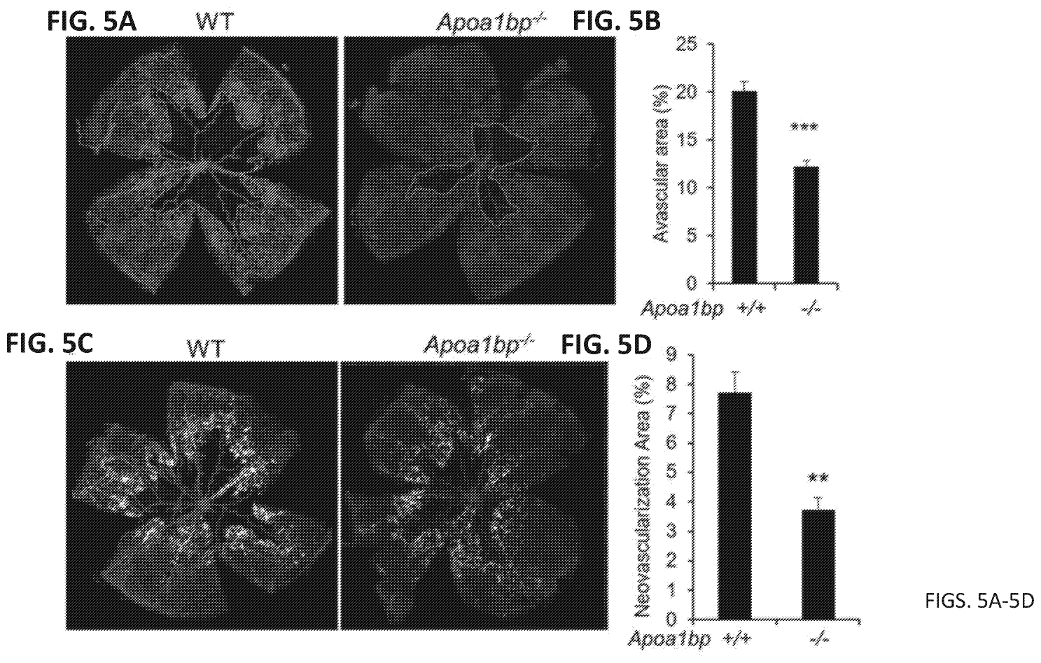

To determine the role of AIBP in OIR, the inventors subjected WT and Apoa1bp^{-/-} mice to OIR condition (FIG. 3) and compared their vascular changes. There was no significant difference in avascular area between WT and Apoa1bp^{-/-} retinas immediately on return to room air at P12, suggesting that AIBP does not protect retinas from vessel loss (FIG. 4). In sharp contrast, there was a significant decrease in avascular area (40% reduction, p<0.001) at P17, indicating a marked increase in reparative angiogenesis in Apoa1bp^{-/-} retinas compared with WT (FIGS. 5A, 5B). Apoa1bp^{-/-} retinas showed a marked reduction (52% reduction, p<0.01) of pre-retinal neovascularization (FIGS. 5C, 5D). Why did AIBP ablation increase reparative angiogenesis but not pre-retinal neovascularization? This is likely due to two reasons: 1) There was more AIBP in the ischemic retinal areas produced by hypoxia retinal neurons than in the vitreous, thus AIBP ablation has a more impact on reparative angiogenesis (which occurs in the retina) than preretinal neovascularization (which occurs toward the vitreous); 2) More rapid restoration of retinal vasculature led to decreased ischemia. The inventors' results indicate an important role of AIBP in impeding revascularization of the ischemic retina between P12 and P17.

### Example 4

### AIBP Overexpression Impeded Reparative Angiogenesis in OIR

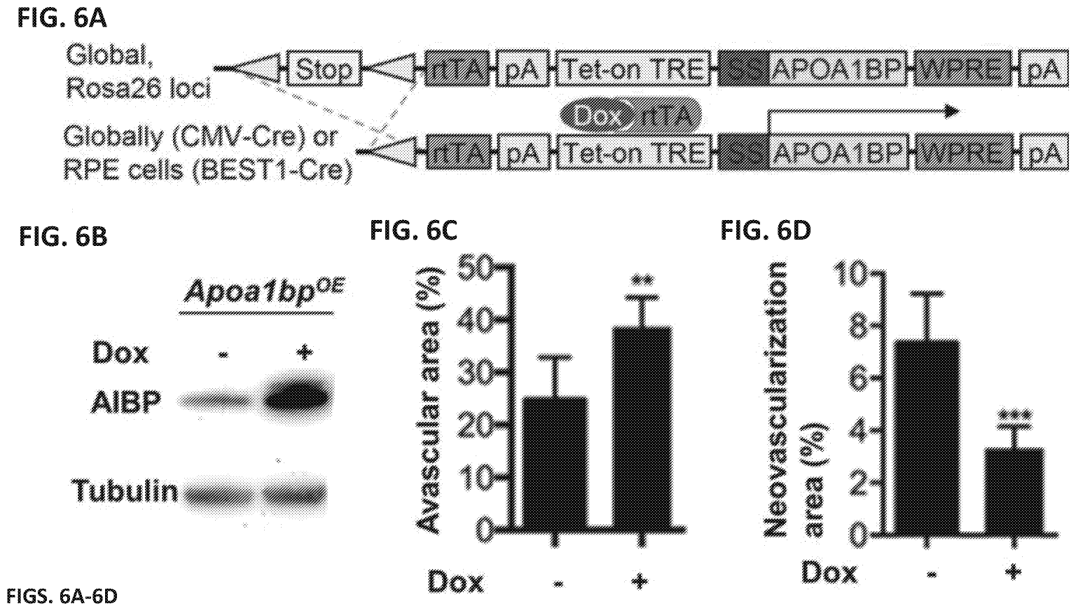

The inventors have generated conditional AIBP overexpression mice (R26Apoa1bp). The transgene, which contains a floxed-STOP cassette, was knocked into the Rosa26 locus (FIG. 6A). The inventors crossed R26Apoa1bp with CMV-Cre (Schwenk et al., 1995) to obtain Apoa1bpOE mice, which enable global doxycycline (Dox)-induced human AIBP expression. The inventors started induction of AIBP expression in neonatal Apoa1bpOE mice at P0 by feeding Dox food to the nursing mother following a published procedure (Cawthorne et al., 2007). Without Dox, AIBP expression is comparable to that of WT mice. Dox food feeding for 17 days induced robust AIBP expression (3.2-fold increase) in the retina (FIG. 6B). The retinal vasculature of induced Apoa1bp^{OE} mice was normal compared with uninduced littermates at P7 before the mice were placed into the hyperoxia chamber. To determine the effect of increased AIBP in OIR, the inventors compared Dox-induced with uninduced Apoa1bp^{OE} mice with respect to vascular changes. AIBP overexpression markedly increased the avascular area (57% increase, p<0.01), consistent with the inventors' hypothesis that AIBP suppresses reparative angiogenesis (FIG. 6C). Substantial amount of AIBP caused a 2.2-fold decrease in neovascularization despite the increased avascular area, suggesting that AIBP has dual roles in suppressing both reparative and pathologic angiogenesis (FIG. 6D).

### Example 5

### AIBP Neutralizing Antibodies Effectively Blocked AIBP'S Effect on HRMEC Proliferation

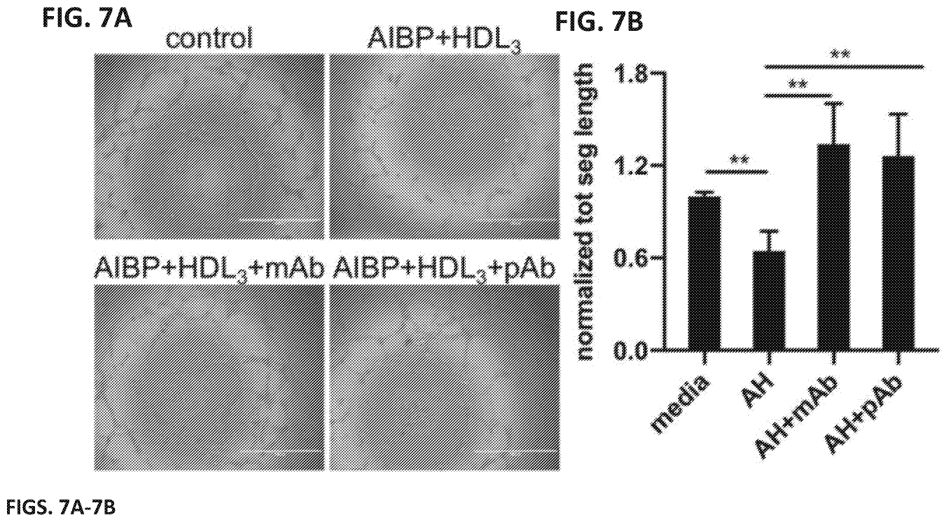

The inventors have shown that AIBP deficiency promoted reparative angiogenesis and reduced preretinal neovascularization in OIR (FIG. 5), suggesting the feasibility to develop a new therapeutic strategy for ROP by neutralizing AIBP. As a proof-of-concept study, the inventors generated a rabbit polyclonal antibody (pAb) and a mouse monoclonal antibody (mAb, 1F9) against AIBP for neutralizing AIBP function. They recognize both mouse and human AIBP proteins, which share 88% amino acid identity. Both antibodies were generated by immunizing animals with recombinant human AIBP proteins. The inventors purified the two antibodies by antigen affinity purification. The inventors validated the specificity of the two antibodies by western blot analysis on mouse retinal lysates (pAb data was shown in FIG. 6B). Preincubation of human AIBP protein with either pAb or mAb abolished the inhibitory effect of AIBP on HRMEC angiogenesis (FIGS. 7A and 7B). Antibody neutralization in HRMECs formed more vascular tubes than in the control media, suggesting that HRMECs may secret AIBP or the media may contain some AIBP. The data suggest that both antibodies are effective in neutralizing extracellular AIBP function.

### Example 6

### APOA1BP^{-/-} Mice Exhibit Normal Retinal Structure and Function

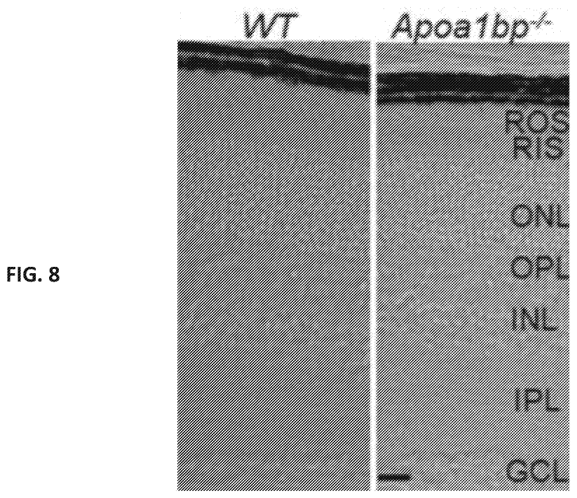

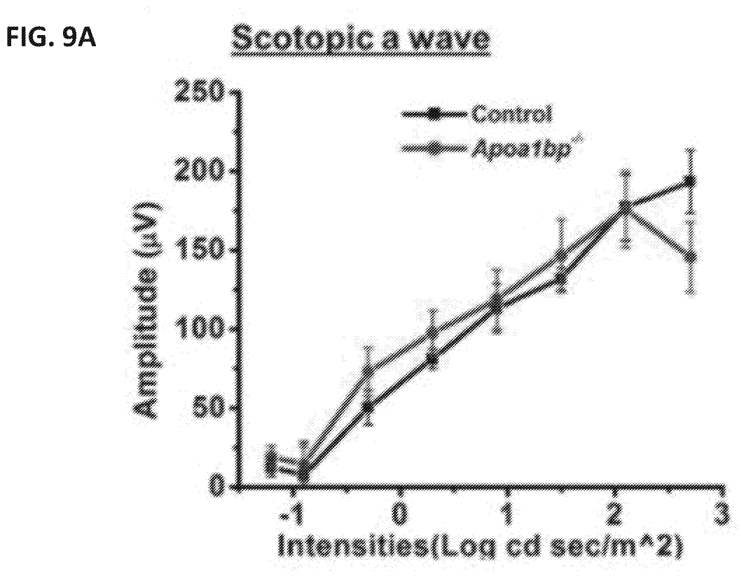

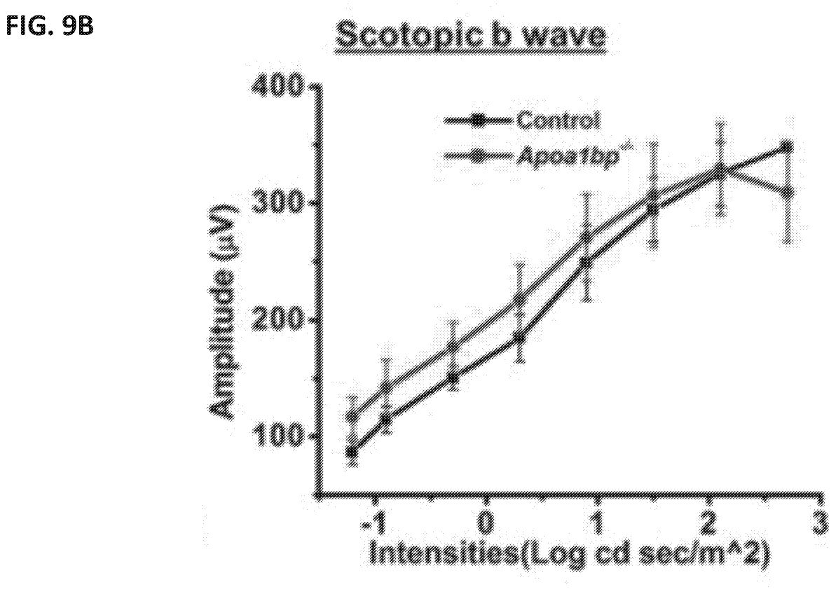

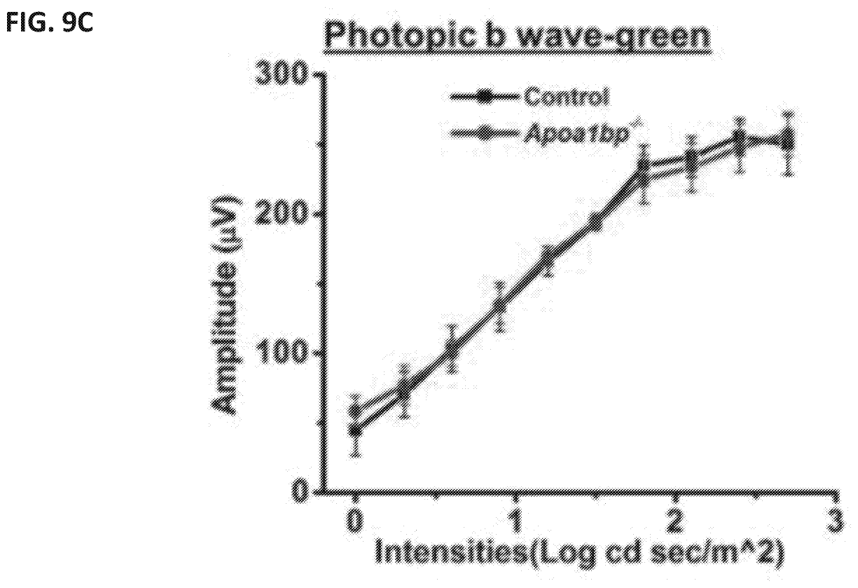

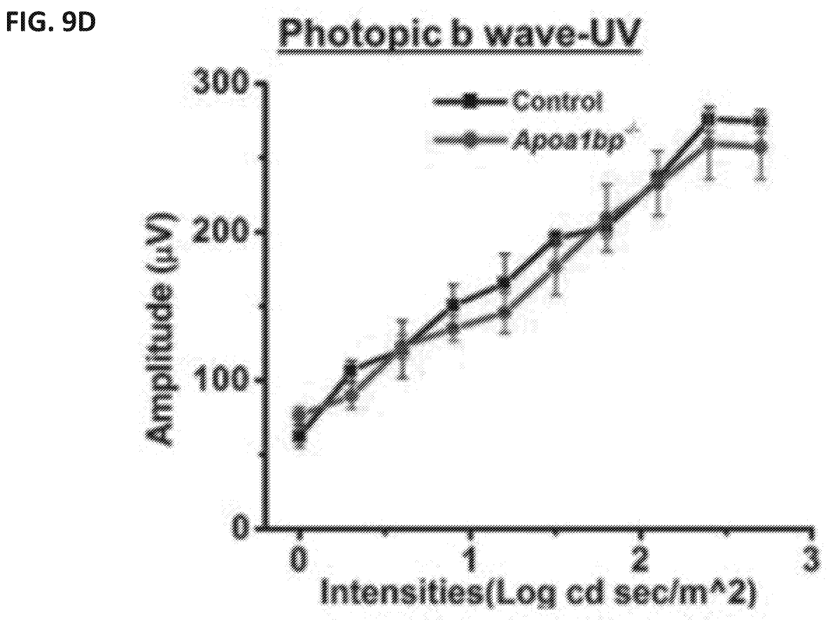

The inventors have generated the Apoa1bp^{-/-} mice, which were viable, fertile, and developed normally (23). Apoa1bp^{-/-} mice (1-month) had normal retinal structure compared with WT (FIG. 8). The inventors measured the scotopic and photopic electroretinogram (ERG) responses of 1-month Apoa1bp^{-/-} and WT mice. Under both conditions, there was no significant difference in both a-wave and b-wave amplitudes under different light intensities (p>0.05) (FIG. 9). Collectively, the inventors' data suggest that AIBP deletion does not impact retinal structure and function (photoreceptors and 2nd order neurons) at this age. This result suggests that targeting AIBP in ischemic retinopathies by neutralizing antibodies is likely to be safe.

### Example 7

### Combination Therapy of AIBP, Apoa-1, and Anti-VEGF Antibody Overcomes the Anti-VEGF Resistance in Experimental Choroidal Neovascularization

Choroidal neovascularization (CNV) causes 80-90% of legal blindness due to age-related macular degeneration (AMD. A significant number of patients are unresponsive to anti-VEGF agents. The mechanisms for anti-VEGF resistance are poorly understood and the effort to develop new treatment is hampered partly due to the lack of suitable AMD animal models that exhibit anti-VEGF resistance. The inventors explored the unique property of the apoA-I binding protein (AIBP) that enhances cholesterol efflux from endothelial cells (ECs) and macrophages, two cell types implicated in CNV, to thereby limit angiogenesis and inflammation to tackle anti-VEGF resistance in CNV. The inventors show that laser-induced CNV in mice with increased age showed increased resistance to anti-VEGF treatment, which correlates with the increased intracellular lipid accumulation in macrophages. The combination of AIBP/apoA-I and anti-VEGF treatment overcomes anti-VEGF resistance and effectively suppresses CNV. Furthermore, macrophage depletion in old mice restores CNV sensitivity to anti-VEGF treatment and blunts the synergistic effect of combination therapy. These results suggest that cholesterol-laden macrophages play a critical role in inducing anti-VEGF resistance in CNV. Combination therapy by neutralizing VEGF and enhancing cholesterol removal from old macrophages is a promising strategy to combat anti-VEGF resistance in AMD.

### AIBP Enhances Cholesterol Efflux and Inhibits Angiogenesis on Retinal and Choroidal ECs.

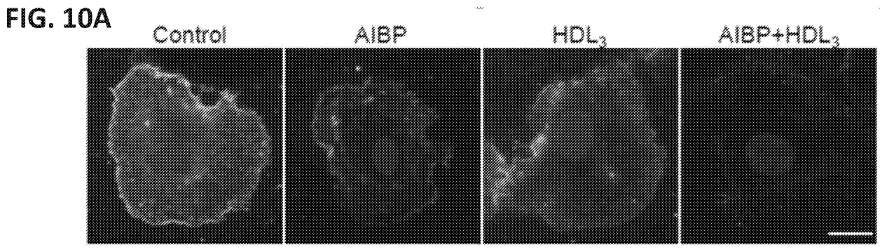

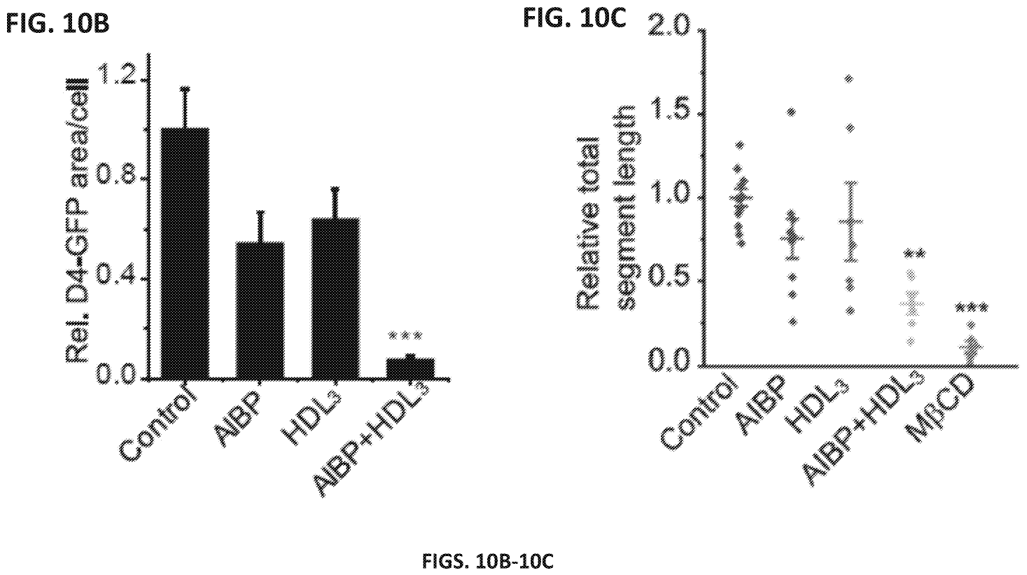

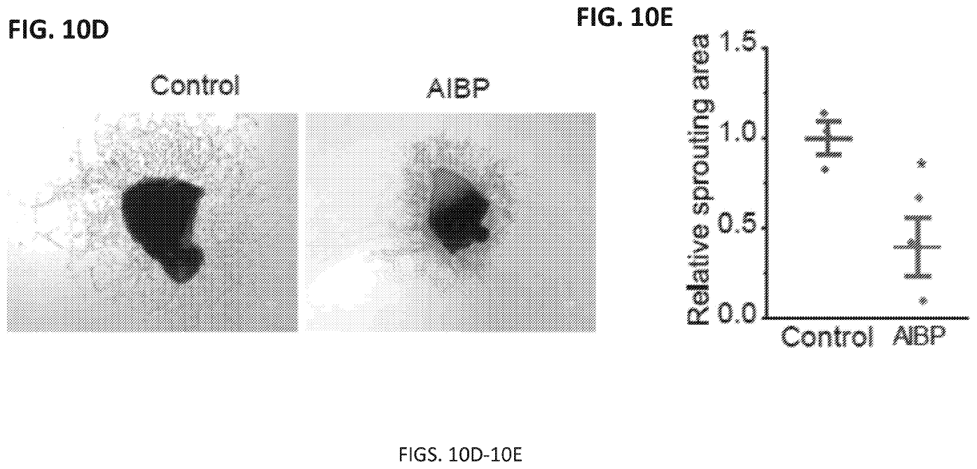

AIBP and HDL together were shown to limit angiogenesis in human umbilical vein ECs (HUVECs) by enhancing cholesterol removal (13). The monkey choroidal EC line RF6/A and human retinal microvascular endothelial cells (HRMECs) are widely used as choroidal and retinal EC models, respectively. Since a recent study showed that the RF6/A cells do not exhibit key EC features (16), the inventors used HRMECs to investigate the role of AIBP in cholesterol efflux in retinal ECs. HRMECs were incubated with control media, AIBP, HDL3 (a subfraction of HDL, which is an efficient cholesterol acceptor) (13), or AIBP in combination with HDL3. The resulting cells were stained with recombinant D4-EGFP, which specifically binds cholesterol and has been used to monitor cellular lipid raft content (17). The incubation with AIBP and HDL3 in combination, but not alone, markedly reduced lipid raft content on the plasma membrane (FIG. 10A, 10B). The data suggest that AIBP regulates cholesterol metabolism in the HRMECs.

The inventors further examined the role of AIBP-mediated cholesterol efflux in HRMEC angiogenesis using the Matrigel-based in vitro tube formation model. As illustrated in FIG. 10C, AIBP and HDL3 cotreatment significantly disrupted in vitro vascular tube formation by HRMECs. As a positive control, cholesterol depletion by methyl cyclodextrin (MβCD) (18,19), a detergent that sequesters free cholesterol, markedly inhibited angiogenesis. To explore the role of AIBP in choroidal angiogenesis, the inventors used the ex vivo model of choroid sprouting, a reproducible model of choroidal angiogenesis (20). Recombinant AIBP potently inhibited choroid sprouting (61% reduction, p=0.035, FIG. 10D, 10E), consistent with the inhibitory role of AIBP in angiogenesis (Note, choroid explants were cultured 4 days in complete EGM-2 media, which contains HDL from bovine serum (21)).

AIBP Reduces Intracellular Lipid Accumulation in Aged Macrophages and Inhibits their Ability to Promote Angiogenesis.

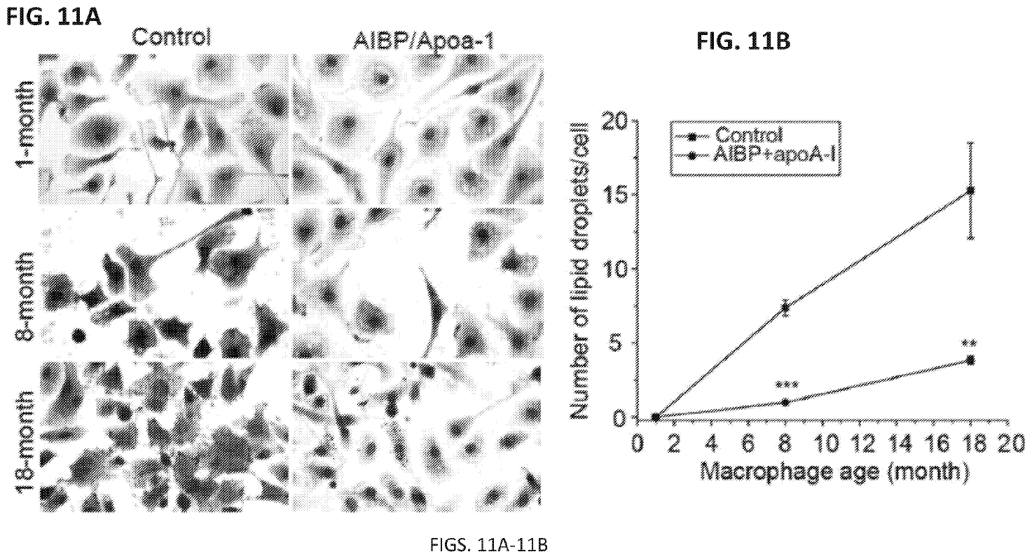

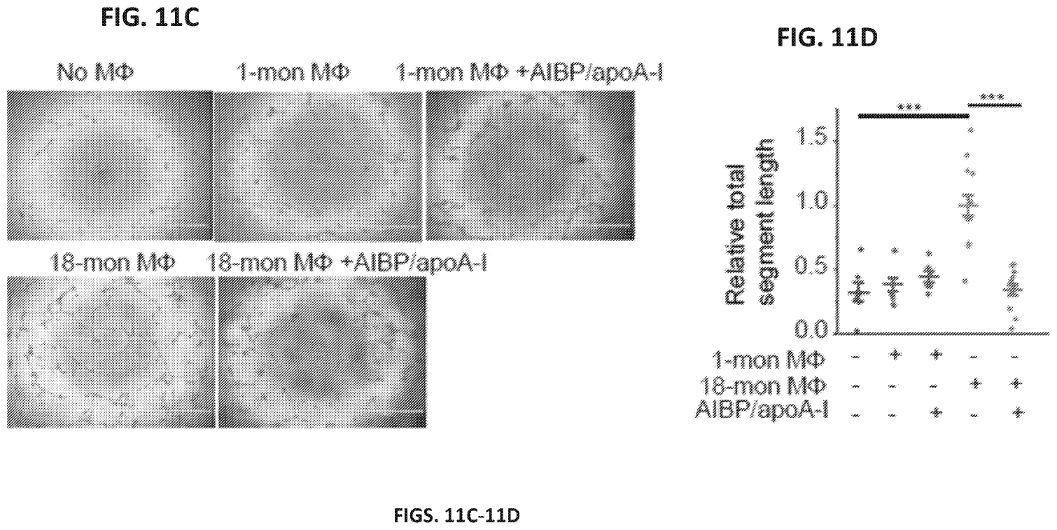

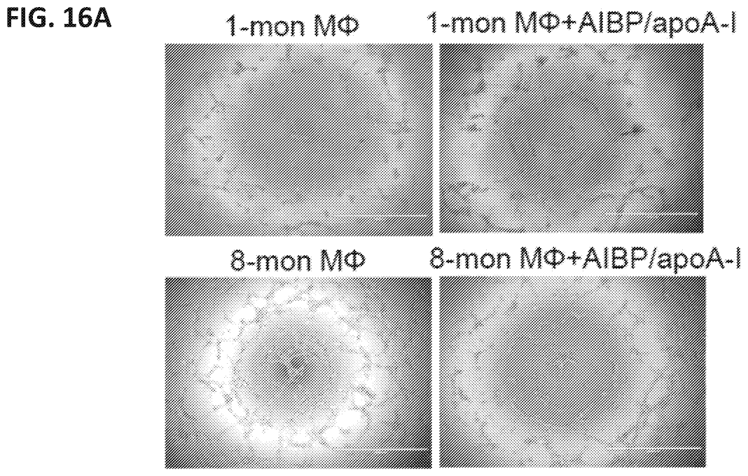

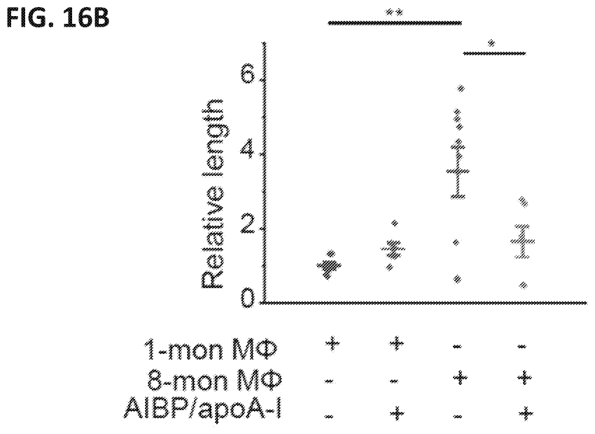

Previous studies have shown that aged macrophages exhibit impaired cholesterol efflux, leading to intracellular lipid accumulation and pathologic vascular proliferation (8). Indeed, the inventors found that peritoneal macrophages isolated from 1-, 8-, and 18-month old mice show an age-dependent increase of intracellular lipids as detected by oil red O staining (FIG. 11A, 11B). Consistent with recent data that AIBP effectively enhances cholesterol efflux from macrophages (14,15,22), AIBP and apoA-I co-treatment markedly reduced lipid accumulation from macrophages that were isolated from 8- and 18-month mice by 86.6% (p<0.001) and 74.9% (p<0.01), respectively (FIG. 11B). Furthermore, AIBP and apoA-I co-treatment significantly inhibited old macrophages' ability to promote angiogenesis of HRMECs co-cultured with peritoneal macrophages from 8-month (FIG. 16) and 18-month (FIG. 11C, 11D) old mice. In contrast, AIBP and apoA-I cotreatment of young macrophages isolated from 1-month old mice had no effect on HRMEC angiogenesis. The inventors' data suggest that AIBP/apoA-I treatment suppresses old macrophages' ability to promote angiogenesis.

### AIBP Deficiency Accelerates Retinal and Choroidal Angiogenesis.

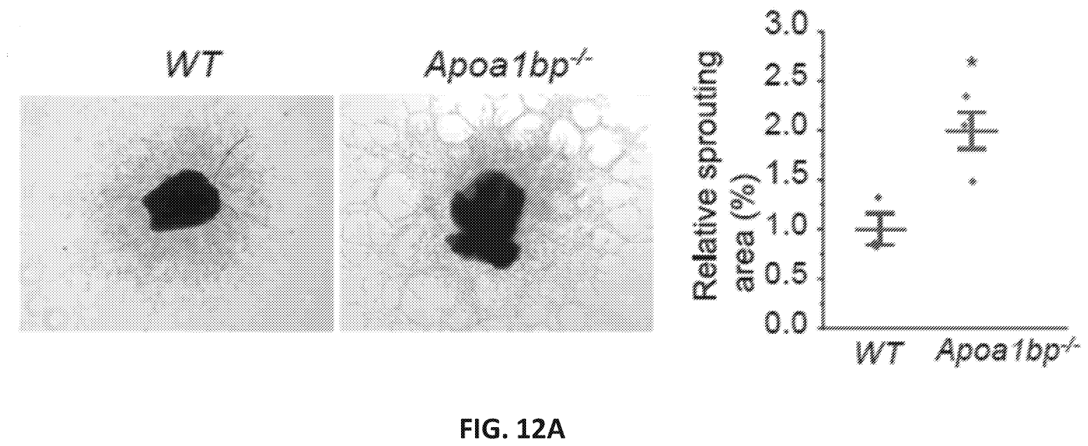

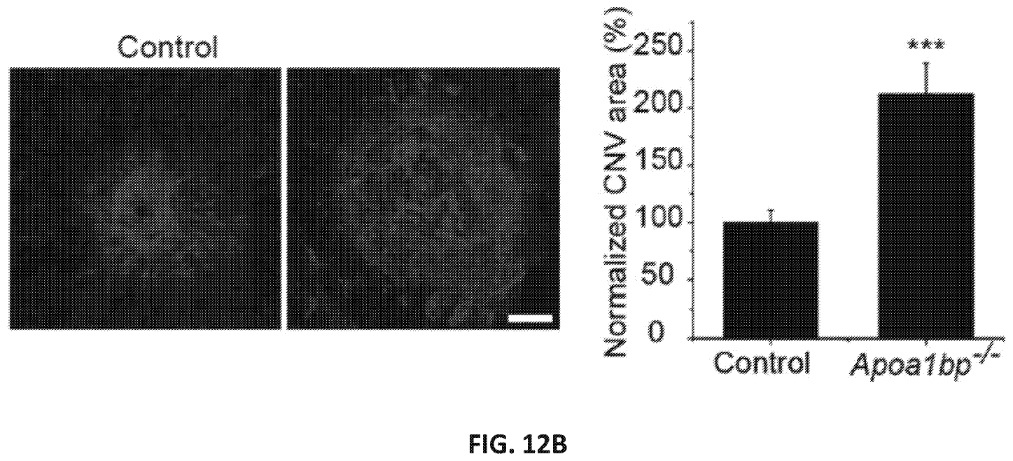

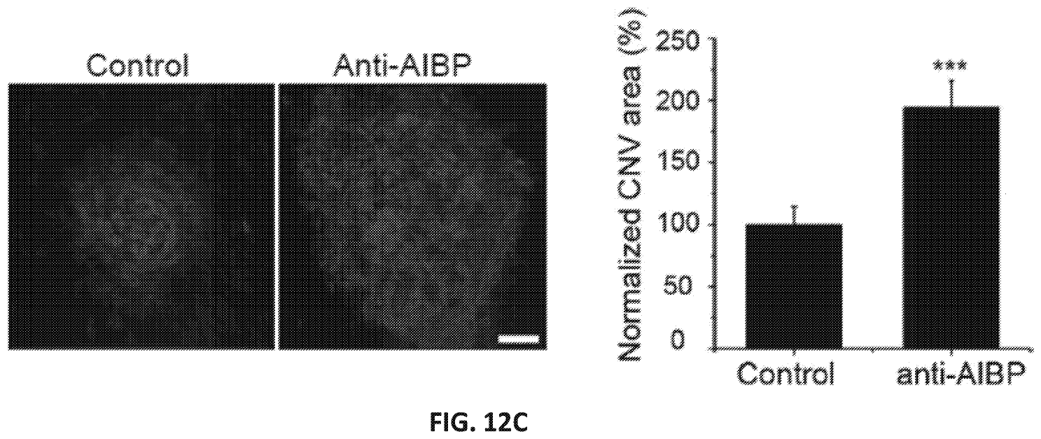

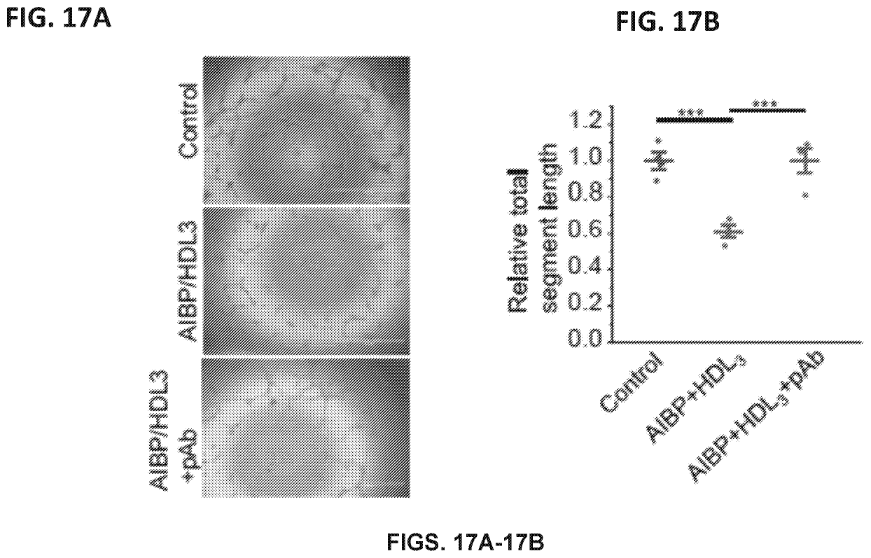

AIBP deficiency was shown to markedly accelerate developmental retinal angiogenesis from P0 to P5 (23). To investigate the role of AIBP on choroid angiogenesis, the inventors compared choroidal microvascular angiogenesis in ex vivo choroid explants isolated from Apoa1bp^{-/-} and WT mice. Apoa1bp^{-/-} choroid explants exhibited a two-fold increase in sprouting area compared with WT controls (p=0.043), consistent with the inhibitory role of AIBP in angiogenesis (FIG. 12A). To examine the role of AIBP in CNV, the inventors induced CNV by laser photocoagulation on WT and Apoa1bp^{-/-} mice (2-3 months) as previously described (24). One week after CNV induction, the CNV area was analyzed by Alexa 568-isolectin labeling on choroidal flatmounts. Loss of AIBP markedly increased the CNV area (2.1-fold, FIG. 12B). To test the inventors' hypothesis that the extracellular AIBP inhibits CNV, the inventors used a rabbit polyclonal antibody (pAb) against AIBP (23) to neutralize extracellular AIBP function. Preincubation of human AIBP protein with pAb abolished the inhibitory effect of AIBP on HRMEC angiogenesis, suggesting that the pAb antibody effectively neutralized extracellular AIBP function (FIG. 17). The inventors delivered 1.3 μg affinity purified pAb by intravitreal injection immediately after laser photocoagulation on WT mice. Consistent with the Apoa1bp^{-/-} data ex vivo (FIG. 12A) and in vivo (FIG. 12B), AIBP neutralization caused a 1.9-fold (p<0.001) increase of the CNV area (FIG. 12C), suggesting that extracellular AIBP inhibits pathogenic angiogenesis.

### AIBP is Reduced in Laser-Induced CNV and Human CNV Specimens.

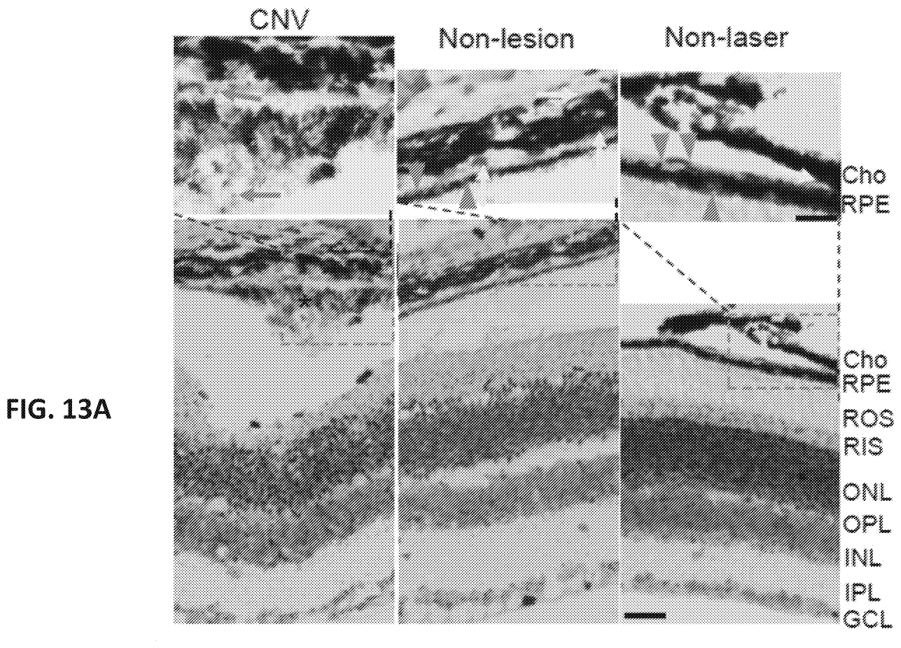

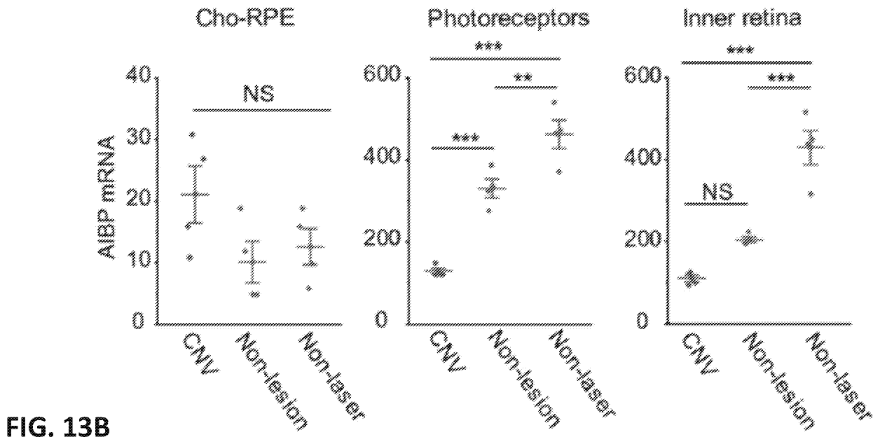

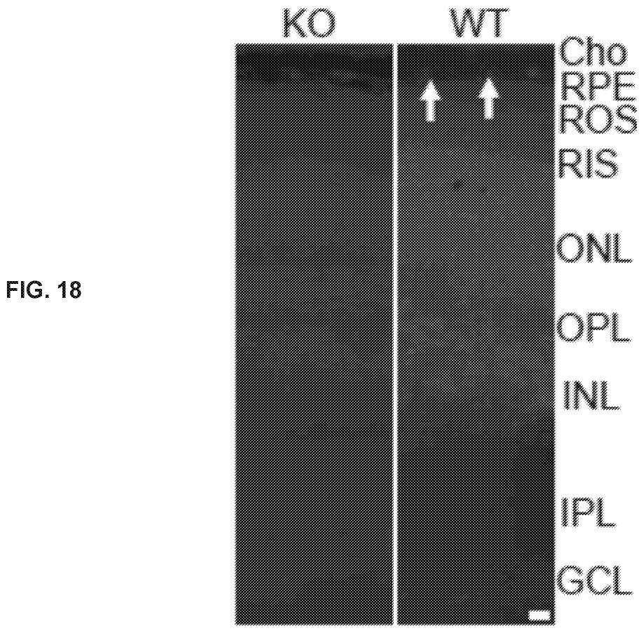

To determine the AIBP expression in laser-induced CNV and human CNV specimens, the inventors used RNAscope, which allows the detection of single mRNA transcripts in intact cells with high specificity (25), to compare AIBP expression in CNV and non-lesion areas adjacent to CNV 7 days after laser injury, and in control retinas not subjected to laser injury (non-laser) in mice. In nonlesion and non-laser controls, AIBP was weakly expressed in the RPE (FIG. 13A, orange arrowheads) and choroid (FIG. 13A, yellow arrows). AIBP mRNA was mainly expressed in photoreceptors (RIS, ONL & OPL) and inner neurons (INL, IPL & GCL). AIBP was also weakly expressed in CNV membranes (FIG. 13A, orange arrows). AIBP expression is not significantly different between CNV, non-lesion and non-laser groups in the choroid-RPE (FIG. 13B). However, AIBP expression in photoreceptors was markedly reduced in CNV compared with both nonlesion and non-laser controls. AIBP expression in the inner retina (INL+IPL+GCL) in CNV was not significantly different from that in non-lesion areas, but was significantly reduced in CNV compared with the non-laser control. In non-lesion areas adjacent to CNV, AIBP in both photoreceptors and inner retina was significantly reduced compared with the non-laser control. The likely reason for AIBP reduction in the non-lesion area is that laser induced CNV causes inflammation and other responses in the adjacent non-lesion areas. Photoreceptors produce the majority of AIBP in the outer retina (more than 20 times than that in RPE/choroid in normal, FIG. 13B), suggesting that the major source of AIBP in the outer retina that suppresses laser-induced CNV is produced by photoreceptors and secreted into the extracellular space. The drastic reduction of AIBP in photoreceptors in CNV contributes to CNV pathogenesis. In the negative control, no specific signal was detected in the Apoa1bp^{-/-} retina, confirming the specificity of the Apoa1bp probe (FIG. 18).

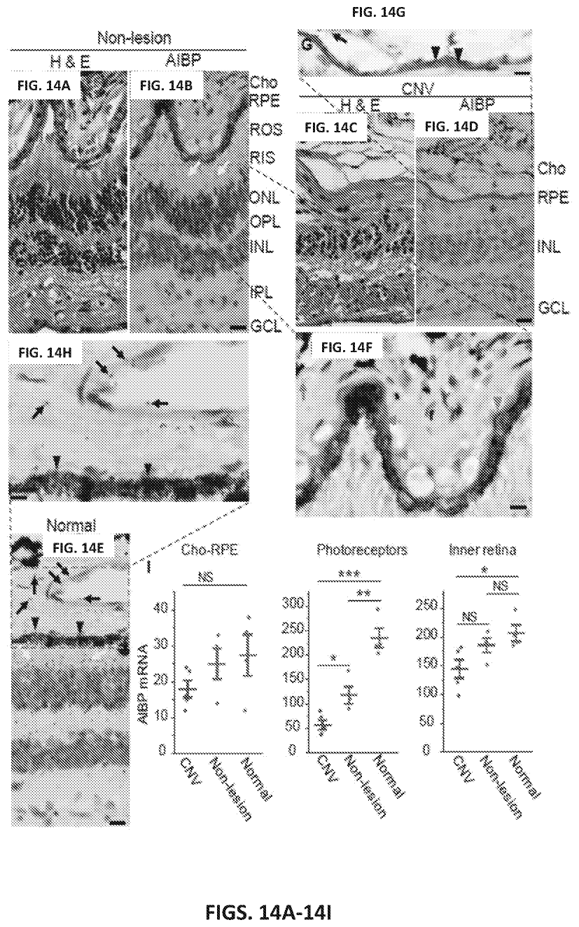

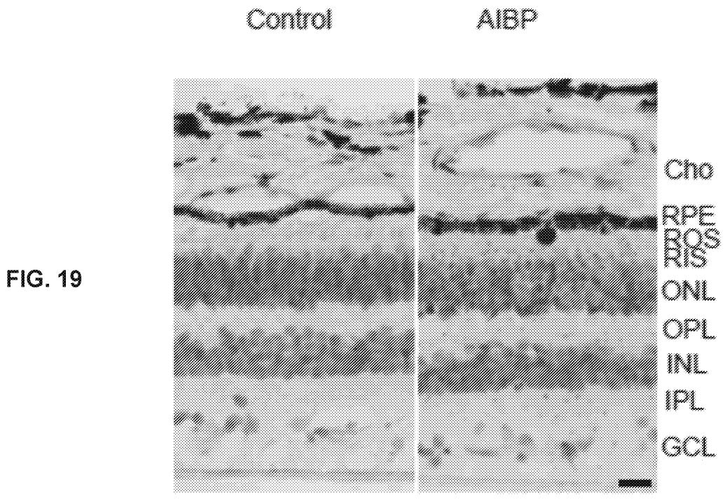

In a parallel study, the inventors examined AIBP expression in human CNV. In normal and non-lesion areas adjacent to human CNV, AIBP was mainly expressed in photoreceptors (inner segment and ONL) and inner neurons (INL, IPL & GCL) (FIG. 14B, 19E, yellow arrows indicating AIBP expression in the inner segment). AIBP was weakly expressed in RPE (FIG. 14B, 14F, orange arrowheads; FIG. 14E, 14H, black arrowheads) and choroid (FIG. 14F, orange arrows; FIG. 14H, black arrows). This expression pattern is similar to that in the mouse retina (FIG. 13A). In CNV lesion areas, H & E staining revealed subretinal disciform scar and loss of photoreceptors and RPE over the scar (FIG. 14C, 14D, asterisk). AIBP was weakly expressed in choroid (FIG. 14G, black arrow) and degenerating RPE (FIG. 14G, black arrowheads). AIBP expression in choroid-RPE is not significantly different between CNV, non-lesion and normal groups (FIG. 14I). AIBP expression in photoreceptors was markedly reduced in CNV area (— 76% reduction, p<0.001) compared with normal. Since the AIBP reduction could be due to photoreceptor loss, the inventors compared AIBP expression in non-lesion areas adjacent to CNV but with relatively intact photoreceptors with that in normal areas. AIBP expression was reduced by 51% (p<0.05) even in non-lesion photoreceptors (FIG. 14I). Thus, AIBP reduction in photoreceptors of CNV lesion is the net result of photoreceptor loss and reduced expression, which is similar to that in laser-induced CNV (FIG. 13A). AIBP expression in the inner retina in CNV was not significantly different from that in non-lesion areas, but was significantly reduced in CNV compared with the normal (FIG. 14I). A negative control using a bacterial probe shows no signal (FIG. 19). Since AIBP deficiency dramatically increases laser-induced CNV (FIG. 12B, 12C), significant AIBP reduction in the outer retina overlying CNV lesions is expected to exacerbate CNV.

### AIBP is Superior to Anti-VEGF Agents at Inhibiting Laser Induced-CNV in 8-Month Old Mice.

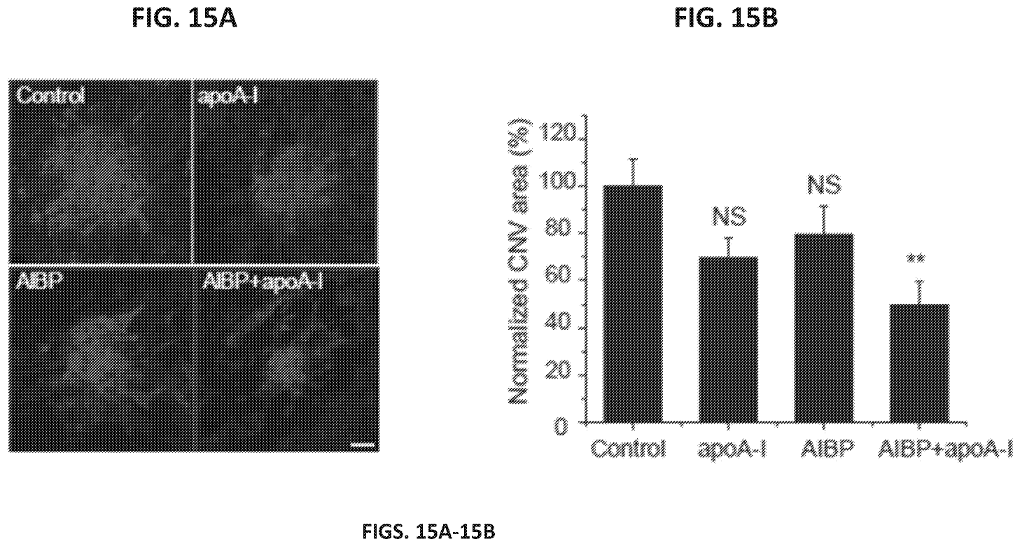

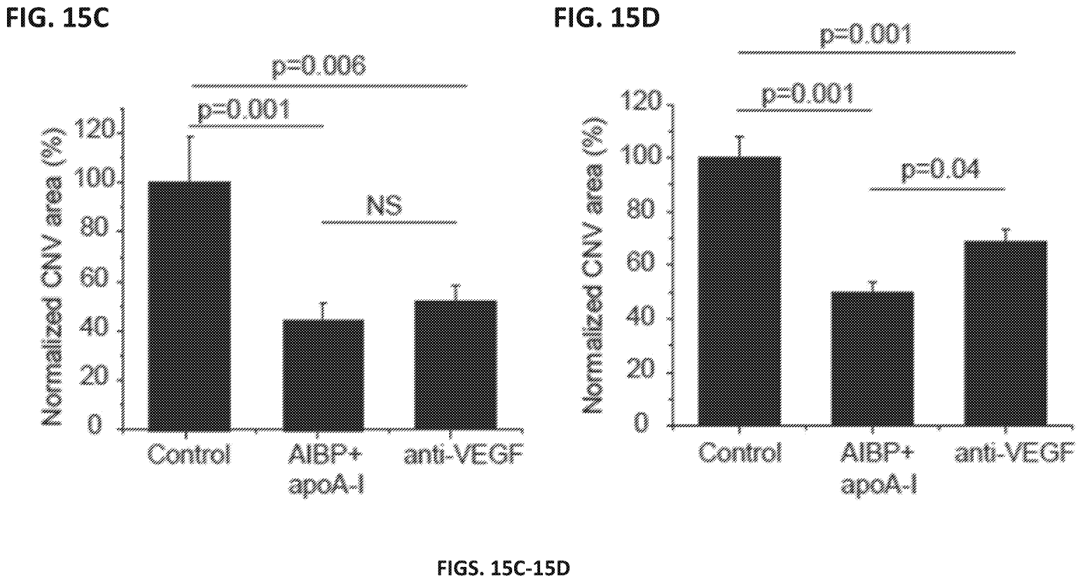

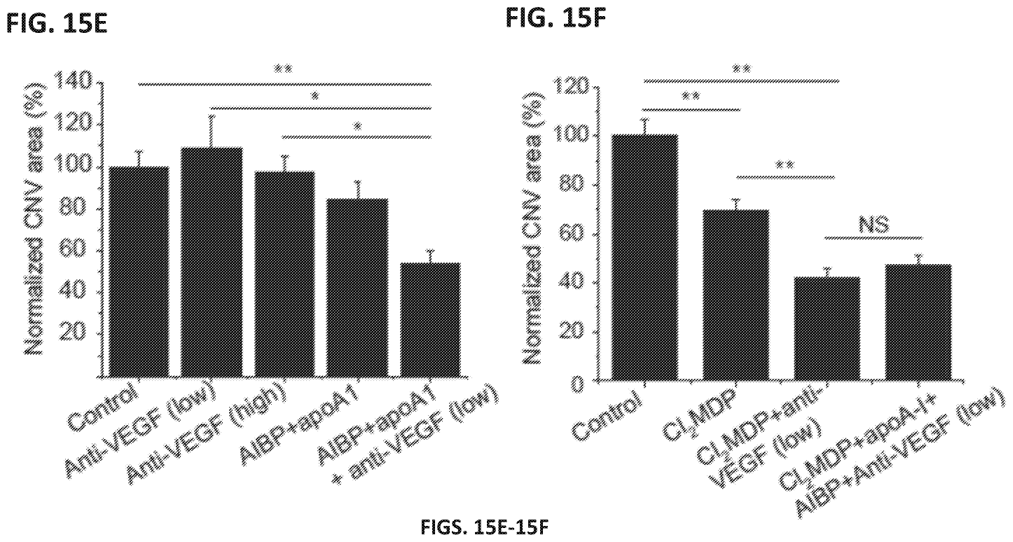

To test the efficacy of AIBP in treating CNV in vivo, the inventors induced CNV in both eyes of WT mice (8-10 weeks) by laser photocoagulation. Immediately after laser delivery, mice received an intravitreal injection of AIBP alone, apoA-I alone, AIBP plus apoA-I, or BSA protein control. Seven days later, the eyes were collected and the CNV area was quantified in choroidal flatmounts. AIBP/apoA-I treatment reduced CNV area by 50.2% (p<0.01) while AIBP or apoA-I alone non-significantly reduced CNV area by 21% and 31%, respectively (FIG. 15A, 15B). These results are consistent with the inventors' in vitro data showing that both AIBP and ApoA-I are necessary to inhibit angiogenesis (FIG. 10C).

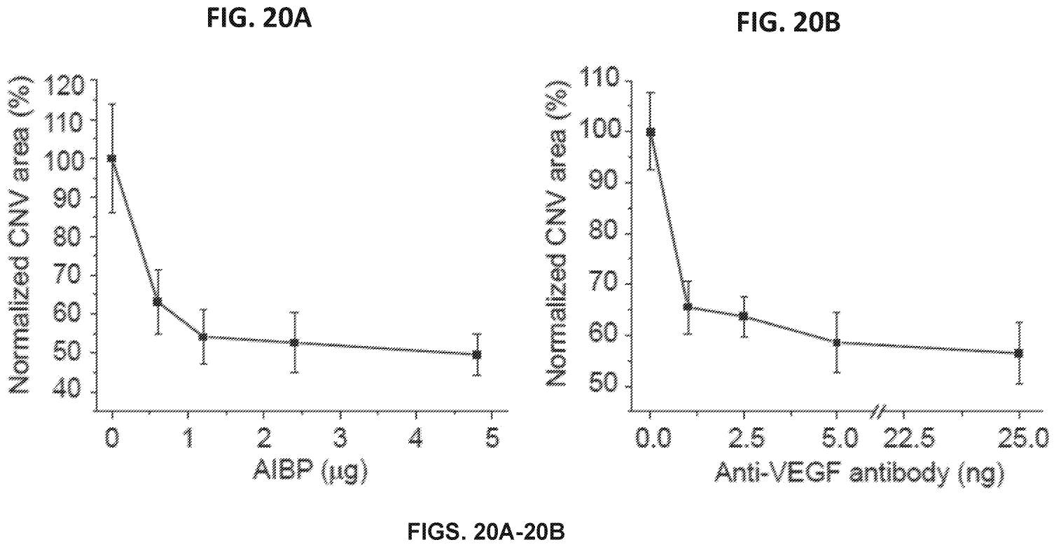

To determine the optimal inhibiting dose of AIBP, the inventors delivered different intravitreal injection doses of AIBP (0.6 μg, 1.2 μg, 2.4 μg, 4.8 μg) in combination with apoA-I (AIBP to apoA-I ratio was kept at 1 μg:4.2 μg) into the eyes of WT mice following the induction of CNV by laser photocoagulation. AIBP and apoA-I co-treatment inhibited laser-induced CNV in a dose-dependent manner (FIG. 20A). The inventors found that the 2.4 μg AIBP and 10 μg apoA-I combination was sufficient to produce maximal inhibition. To compare the efficacy of AIBP with anti-VEGF treatment, the inventors first determined the dose-response curve of an anti-VEGF antibody (AF-493-NA, R&D Systems) given by intravitreal delivery at inhibiting laser-induced CNV. This anti-VEGF antibody dose-dependently inhibited CNV, and that 5 ng anti-VEGF antibody achieved maximal inhibition (FIG. 20B). This amount was used to compare with that of the AIBP treatment (2.4 μg AIBP and 10 μg apoA-I combination). AIBP/apoA-I was equally effective as the anti-VEGF antibody at inhibiting laser-induced CNV in young mice (6-8 weeks) (FIG. 15C). Since AIBP targets both hyperactive VEGFR2 signaling in choroidal ECs and intracellular lipid accumulation in old macrophages, two processes implicated in CNV pathogenesis, the inventors tested the hypothesis that AIBP is more effective than anti-VEGF agents in inhibiting CNV in older mice. Indeed, AIBP/apoA-I treatment reduced CNV area more than the anti-VEGF antibody in 8-month mice (49.9% vs. 68.4% CNV area, p=0.04) (FIG. 15D). This is primarily due to the decreased efficacy of anti-VEGF antibody compared with that in young mice (68.4% vs. 51.8% CNV area).

### AIBP/apoA-I and Anti-VEGF Combination Therapy Overcomes Anti-VEGF Resistance in Treating Laser-Induced CNV in Old Mice.

CNV is a process that involves both angiogenesis and inflammation (Campa et al., 2010). Reduced cholesterol efflux in old macrophages (Sene et al., 2013) and local inflammation are implicated in CNV, which may contribute to anti-VEGF resistance. Since AIBP has the ability to enhance cholesterol efflux from old macrophages and reduce inflammation (Schneider et al., 2018; Zhang et al., 2016, 2018), the inventors explore the potential to use AIBP in combination with the anti-VEGF reagents to address the anti-VEGF resistance issue. For this purpose, the inventors established an AMD animal model for anti-VEGF resistance by performing laser induced CNV on aged mice (18-month old). The reason is that old macrophages in aged mice accumulate large amount of cholesterol in comparison to young macrophages due to impaired cholesterol efflux and promote CNV formation (Sene et al., 2013). Macrophages played an important role causing resistance to anti-VEGF therapy in cancer patients (Dalton et al., 2017). In addition, aged mice have increased inflammatory responses compared with young mice after the laser CNV procedure. The inventors performed laser CNV on 18-month male C57BL/6J mice. Male mice were used because old female mice showed more variability in CNV size after laser CNV (Espinosa-Heidmann et al., 2002, 2005).

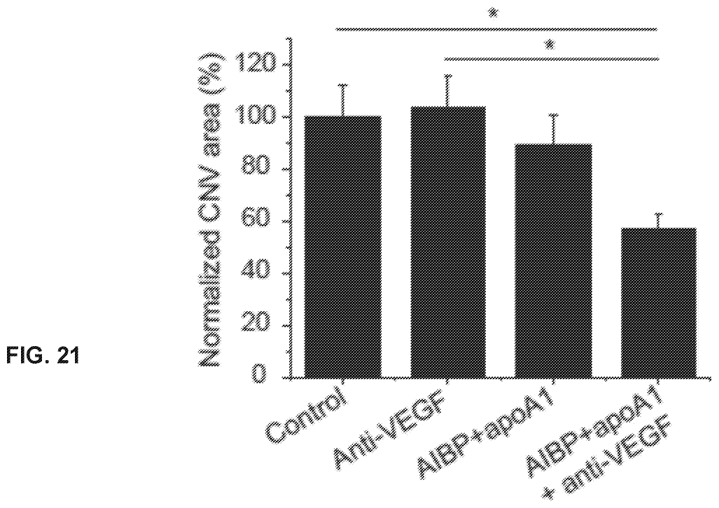

The inventors have shown age-dependent increase of intracellular lipids in macrophages (FIG. 11A, 11B). The inventors thus expected that AIBP/apoA-I would be more effective than the anti-VEGF antibody in suppressing laser-induced CNV in 18-month than in 8-month old mice. However, neither AIBP nor anti-VEGF treatment was effective in inhibiting CNV in 18-month mice (FIG. 15E). No inhibition was observed even after the inventors increased the amount of anti-VEGF antibody by five times (high, 25 ng) (FIG. 15E), which suggests that old mice are resistant to anti-VEGF treatment. Remarkably, the combination of AIBP/apoA-I and anti-VEGF (low, 5 ng) antibody overcame the anti-VEGF resistance and robustly suppressed laser induced CNV (46.5% reduction, p<0.001). The inventors observed a similar effect by AIBP/apoA-I and anti-VEGF antibody in old female mice (14-15 months) (FIG. 21). The synergistic effect is likely due to AIBP's ability to disrupt VEGF-independent angiogenic pathways by regulating lipid rafts in ECs and macrophages (see more below).

### Macrophage Depletion in Old Mice Restored CNV Sensitivity to Anti-VEGF Treatment and Blunted the Synergistic Effect of Combination Therapy.

The anti-VEGF resistance is likely caused by cholesterol-laden macrophages in old mice. To test this hypothesis, the inventors used clodronate liposomes (C12MDP) to deplete macrophages in 18-month mice and performed laser-induced CNV. Macrophage depletion led to a 31% reduction in CNV lesion size, consistent with previous studies (FIG. 15F) (9,10). Notably, the anti-VEGF antibody became effective at inhibiting CNV after macrophage depletion, suggesting that cholesterol laden old macrophages play a key role in conferring anti-VEGF resistance in old mice. Furthermore, combination therapy of AIBP/apoA-I with the anti-VEGF antibody did not exhibit any synergistic effect after macrophage depletion (FIG. 15F). This experiment suggests that AIBP/apoA-I treatment augments the effectiveness of anti-VEGF therapy by targeting old macrophages. The most parsimonious explanation is that AIBP enhances cholesterol removal from old macrophages, which inhibits their ability to promote pathogenic angiogenesis (see FIG. 11C, 11D).

### Discussion

Anti-VEGF resistance remains a major challenge to current anti-VEGF therapy for CNV. Various strategies have been tested to overcome this issue, including increasing the frequency of anti-VEGF therapy, switching to different anti-VEGF agents, and combining anti-VEGF therapy with another treatment modality, e.g., photodynamic therapy. Various combination therapies are currently explored in clinical trials, e.g., targeting PDGF (Fovista) or the angiopoietin pathway. However, no major breakthrough has been reported. In fact, a phase III trial combining anti-VEGF and PDGF failed to demonstrate improved efficacy.

The mechanisms for anti-VEGF resistance remains elusive and the effort to develop new treatment is hampered in part, due to the lack of suitable AMD animal models that exhibit anti-VEGF resistance. By using mice from different ages, the inventors demonstrated that laser-induced CNV in mice with increased age showed increased resistance to anti-VEGF treatment. In young mice (6-10 weeks), the most widely used age group due to their consistency and lack of variability in laser-induced CNV (24), AIBP was equally effective as an anti-VEGF neutralizing antibody in suppressing laser-induced CNV. In the middle-aged group (8-10 months), the anti-VEGF agent was less effective whereas AIBP maintained the same efficacy as in treating the young mice. In old mice (>18 months), neither AIBP nor the anti-VEGF agent was effective in suppressing CNV. Remarkably, the combination of AIBP and anti-VEGF treatment overcomes the anti-VEGF resistance and effectively suppresses CNV. Several lines of evidence suggest that the accumulation of intracellular lipids in old macrophages plays a critical role in anti-VEGF resistance in this model. First, the decrease in efficacy of anti-VEGF therapy with age is inversely correlated with the age-dependent increase of intracellular lipids in macrophages. Second, macrophage depletion in old mice restores CNV sensitivity to anti-VEGF treatment. Third, the beneficial effect of AIBP is likely due to both its ability to enhance cholesterol efflux from macrophages and its anti-inflammatory function (14,15,22). Further studies are necessary to determine the molecular mechanism(s) connecting aged macrophages and anti-VEGF resistance.

CNV is a process that involves both angiogenesis and inflammation (26). The inventors' data suggest that the VEGF-dependent angiogenic pathway plays a dominant role in CNV pathogenesis in young mice (6-10 weeks). However, with aging, alternative angiogenic pathways involving cholesterol-laden macrophages and ECs exert increasingly larger roles in CNV, leading to resistance to anti-VEGF monotherapy. Previous studies have shown that VEGF165 acts as a proinflammatory cytokine targeting monocytes, macrophages, and leukocytes in a positive feedback loop involving primarily ECs to sustain pathological neovascularization (11,12). This may explain why both the neutralization of extracellular VEGF by anti-VEGF agents and the enhanced removal of cholesterol from old macrophages by AIBP/apoA-I are required to cut off the vicious cycle between ECs and macrophages to combat anti-VEGF resistance. Furthermore, both Apoa1bp^{-/-} and AIBP neutralization data showed that AIBP plays a critical role in regulating pathogenic angiogenesis (i.e. CNV) likely by enhancing cholesterol efflux from both ECs and macrophages (FIG. 12B, 12C). Significant AIBP reduction in the outer retina in human CNV lesions (FIG. 14) is expected to exacerbate CNV in human neovascular AMD, and contribute to CNV pathogenesis. The inventors speculate that AIBP in the outer retina, which is mainly produced by photoreceptors, plays an important role in inhibiting the progression of choroidal NV in subretinal space while AIBP in both inner and outer retina may play a role in inhibiting type 3 NV. Thus, delivery of exogenous AIBP/apoA-I is a novel treatment that could reduce CNV or overcome anti-VEGF resistance for patients with neovascular AMD.

Substantial evidence indicate that macrophages have an important role in the pathogenesis of wet AMD in both animal models and human patients (8-10, 27-33). Oxidized low-density lipoprotein and macrophages have been detected in CNV membranes from eyes with AMD (34). In particular, macrophage density and the proliferation of infiltrated inflammatory cells are significantly increased in CNV membranes from patients previously treated with Avastin (35), which implies a mechanism for tachyphylaxis to anti-VEGF treatment. The inventors' study provides strong evidence that cholesterol-laden macrophages confer anti-VEGF resistance in wet AMD and that combination of anti-VEGF agents and AIBP/apoA-I can be a potential therapeutic solution. Since anti-VEGF therapy has become the mainstay of treatment for a plethora of ocular pathologies including CNV, diabetic retinopathy, and retinal vein occlusion, etc., this work will have broad implications in the clinic in treating patients that are not benefiting from the current therapy. Although the laser-induced model of CNV does not have the age-related progressive pathology in AMD, it captures many of the important features of the human condition (e.g., newly formed vessels arise from the choroid and invade into the subretinal space, accumulation of macrophages near arborizing neovascular membranes (36-39, etc.). This model has been successful in predicting the clinical efficacy of anti-VEGF therapy for neovascular AMD (40). It is also widely used for studying CNV and assessing anti-angiogenic drugs in vivo. The involvement of macrophages in anti-VEGF resistance in this model and in human AMD (35) suggest the validity of this model to study the mechanism and treatment strategies of anti-VEGF resistance. Since there are no animal models recapitulating all features of neovascular AMD, the efficacy of AIBP/apoA-I/anti-VEGF combination therapy in overcoming anti-VEGF resistance in human AMD ultimately needs to be evaluated in human clinical trials.

### Example 8

### Estimation on the Clinical Dose of AIBP, APOA-I, and Anti-VEGF Antibody in Treating CNV in Human

The human vitreous volume (5.2 mL) 1 is ˜1000 times that of the mouse vitreous volume (5.3 μL) (2). Based on the vitreous volumes, the inventors estimated that optimal dose for AIBP and apoA-I are 2.4 mg AIBP and 10 mg apoA-I (dose range, 1.2 mg-4.8 mg AIBP, 5.0 mg-20 mg apoA-I). The optimal dose for anti-VEGF antibody is 5 mg, which is close to the 1.25 mg typical dose for Avastin in treating CNV patients.

### Example 9

### Examples of Materials and Methods

### Mice

WT (C57BL/6J) mice were purchased from Jackson Laboratory. Old male C57BL/6 mice (18 months) were ordered from Jackson Laboratory or National Institute of Aging. Old female C57BL/6 mice (14-15 months) were ordered from the Comparative Medicine of Baylor College of Medicine or bred from Jackson mice. Apoa1bp^{-/-} mice were generated previously (23). All animal experiments were approved by the Institutional Animal Care and Use Committees (IACUC) at Baylor College of Medicine, Houston, and Houston Methodist Research Institute, Houston.

### Cells

Human retinal microvascular endothelial cells (HRMECs) were purchased from Cell Systems Corporation (Kirkland, Wash., USA), and cultured at 37° C. with 5% CO₂ in a humid atmosphere in Endothelial Basal Medium (EBM-2) with 2% fetal bovine serum (FBS). Peritoneal macrophages recruitment was elicited by intraperitoneal injection of 4% thioglycollate. Five days after injection, macrophages were harvested and cultured in DMEM/F12 with 10% FBS, 100 U/mL penicillin, and 100 μg/mL streptomycin at 37° C. overnight (41). Macrophages were then washed with PBS and non-adherent cells were removed.

### D4-GFP Staining to Visualize Cholesterol in Cellular Membrane

1.5×10⁴ HRMECs were placed on 0.2% gelatin-coated coverglasses in a 24-well plate. On the following day, HRMECs were treated with control base medium, recombinant AIBP (100 ng/mL), HDL3 (50 m/mL), or combination (100 ng/mL AIBP and 50 μg/mL HDL3) conditions in EBM-2 supplemented with 0.1% BSA for 4 hours in a 37° C. incubator. After washing with PBS for 3 times, cells were incubated with recombinant D4-GFP (75 μg/mL) in complete EBM-2 for 1.5 hours on ice. After washing with cold PBS 3 times, cells were fixed with 4% paraformaldehyde 10 min at room temperature and mounted for imaging with a Leica epifluorescence microscope (Leica DM4000). GFP positive areas per cell were quantified using ImageJ.

### Tube Formation Assay

HRMECs were seeded in each well of a 96-well plate, which were coated with 1:1 mix of Matrigel (Corning, USA) and EBM-2, at a density of 1×10⁴ cells/well in 200 μL EBM-2. For HRMEC-macrophage co-culture, peritoneal macrophages were preincubated with 0.2 μg/mL AIBP and 25 μg/mL apoA-I (individually or in combination) for 4 hours (23). HRMECs and peritoneal macrophage were then mixed (10:1 ratio) and seeded on growth factor-reduced Matrigel mixed 1:1 with EBM-2 in 96-well culture plate. Cells were incubated at 37° C. for 4-6 hours before being imaged by a light microscope. The total segment length or tube length was quantified using ImageJ.

### Choroidal Sprouting Assay

Choroidal sprouting experiments were performed as previously described 20. The RPE/choroid from 4-week old C57BL/6J or Apoa1bp^{-/-} mice were cut into approximately 1×1 mm² pieces, and placed in growth factor-reduced Matrigel in 24-well plates with endothelial cell ECM complete medium at 37° C. For explants treated with AIBP/apoA-I, 0.2 μg/mL AIBP and (25) μg/mL apoA-I (alone or in combination) were added to the medium and incubated for 4 hours every 2 days. Images of individual explants were taken 4-5 days after embedding and the area of microvascular sprouting was quantified using ImageJ.

### Oil Red O Staining

Peritoneal macrophages were washed twice with cold phosphate-buffered saline (PBS) and fixed with 4% paraformaldehyde (PFA) for 10 min at 37° C. Cells were placed in 100% propylene glycol and incubated for 10 min at room temperature with occasional shaking. Cells were then incubated with pre-warmed 0.5% oil red O at 65° C. for 10 min with occasional stirring. After removing the oil red O solution, cells were incubated with 75% propylene glycol for 5 min at room temperature, washed with H2O, and counterstained with hematoxylin prior to examination by microscopy.

### Laser-Induced Choroidal Neovascularization (CNV)

Laser photocoagulation was carried out as described previously using the Micron IV retinal imaging system (Phoenix Research Lab, Pleasanton, Calif., USA) with the Meridian Merilas 532 green laser (240 mW, 200 ms, 50 μm spot) (24). C57BL/6J mice (males and females: 6-8 weeks and 2-3 months; 8-10-month-old males, 18-month-old males, and 14-15-month-old females) and Apoa1bp^{-/-} mice (2-3 months, males and females) were used. Slightly younger female mice were used in the old mouse group because old female mice showed more severe CNV than males (42,43). At 1 week after laser treatment, CNV was analyzed in choroidal flat mounts by Alexa 568 isolectin B4 staining and lesion size was quantified by ImageJ.

### Intravitreal Delivery

Intravitreal injection in mice was performed as previously described (44) with an injection volume of 1.2 pt. For most experiments, 2.4 μg AIBP, 10 μg apoA-I, 5 ng anti-VEGF antibody (AF-493-NA, R&D Systems), 12.4 μg BSA, and 5 ng purified goat IgG (control for anti-VEGF antibody) were delivered individually or in combination unless otherwise indicated. For antibody neutralization of AIBP, 1.3 μg affinity purified rabbit anti-AIBP polyclonal antibody (23) was delivered by intravitreal injection immediately after laser photocoagulation to WT mice.

### Human Specimens

Human CNV specimens were obtained from three patients: one 75-year-old man, one 80-year old man, and one 90-year-old man. All patients were of Caucasian ethnicity with neovascular AMD. Three control eye specimens were obtained from 57-80-year-old Caucasian donors without AMD. The use of the postmortem human donor eyes was approved by the Institutional Review Board (IRB) at Baylor College of Medicine, Houston, and Johns Hopkins University School of Medicine, Baltimore.

### RNAscope

Retina sections of human or mouse eyes were used for RNAscope assay to detect AIBP mRNA expression and localization. Tissues were hybridized with target oligo probes (Advanced Cell Diagnostics, Newark, Calif.) for mouse or human AIBP, or a negative control probe targeting bacterial dihydrodipicolinate reductase. The AIBP was detected with the RNAscope Fluorescent Multiplex Kit or RNAscope 2.5 HD Chromogenic Detection Kit (Advanced Cell Diagnostics) according to the manufacturer's protocol with the following modifications: 1. For the fluorescent RNAscope assay with mouse eyes, tissues were post-fixed in 4% PFA for 90 minutes at room temperature to preserve tissue integrity after baking slides for 30 min at 60o C; 2. For the chromogenic RNAscope assay with human eyes, target retrieval duration was set at 10 minutes and the amplification step 5 was doubled to 60 minutes. Images were collected with an Olympus BX53 microscope (for Fast Red detection) or Zeiss LSM800 Confocal microscope (for fluorescent detection).

### Macrophage Depletion

Mice were anesthetized by intraperitoneal injection of ketamine/xylazine (70-100/10-20/kg body weight). Splenic and systemic macrophage depletion was performed with 150 μL Clodrosome (18.4 mM CL2MDP, Encapsula NanoSciences LLC, Brentwood, Tenn.) by intraperitoneal (IP) administration 3 days and 24 hours before the laser procedure and 3 days after the laser procedure. Control group received IP administration of PBS.

### Statistical Analysis

All group results are expressed as mean±SEM. Comparisons between groups were made using the two-tailed Student's t-test or one-way ANOVA and Tukey's post hoc or Kruskal-Wallis tests for multiple groups. Levene's test was used to access homogeneity of variance. Values of ‘N’ were described in figure legends. Statistical significance was denoted with *P<0.05, **P<0.01, ***P<0.001 in the figures and figure legends. Statistical analysis was performed with OriginPro or GraphPad Prism.

### Example 10

### Compositions and Methods for Treating Neovascularization and Ischemic Retinopathies by Targeting Angiogenesis and Cholesterol Transport

Older age and larger CNV lesion at baseline are associated with worse anti-VEGF treatment outcomes from multiple pivotal clinical trials (e.g., ANCHOR, MARINA, CATT)¹⁻⁴. Laser photocoagulation produced larger CNV lesions in old mice, which is much more resistant to anti-VEGF treatment, in comparison with young mice⁵. Furthermore, anti-VEGF resistance in CNV patients is frequently associated with arteriolar CNV, which is characterized by large-caliber branching arterioles, vascular loops and anastomotic connections, but minimal capillary components⁶. Persistent fluid leakage in arteriolar CNV likely occurs because of increased exudation from poorly formed tight junctions at arteriovenous anastomotic loops in the setting of high rates of blood flow. On the other hand, anti-VEGF responders are characterized with capillary CNV, in which leakage occurs as a result of VEGF-mediated permeability in leaky capillaries.

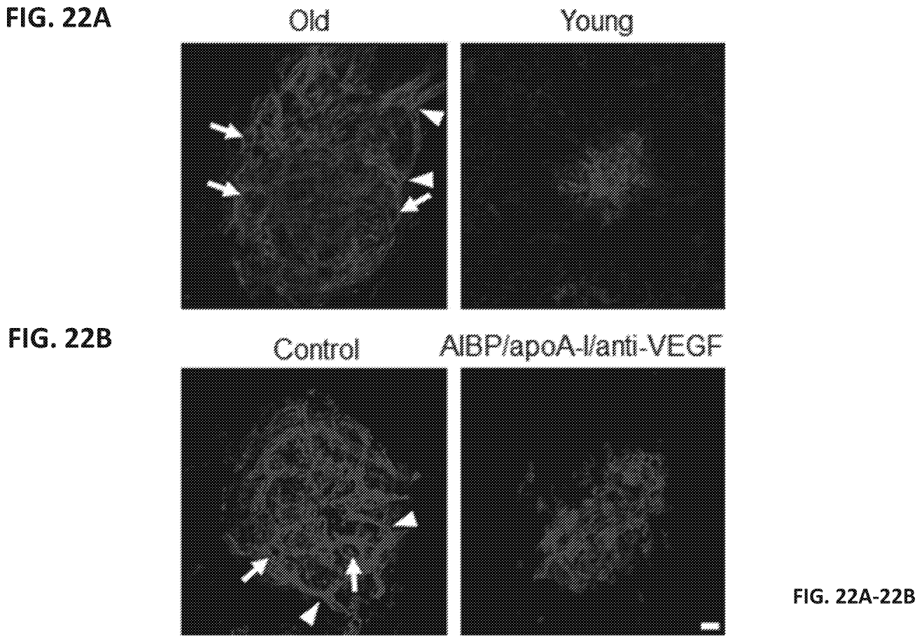

Laser-induced CNV in young (2-month) and old (18-month male or 14-15-month female) mice exhibited similarity to capillary and arteriolar CNV, respectively (FIG. 22A, white arrowheads and arrows indicate branching arterioles and vascular loops in old mice, respectively). The data is consistent with previous work⁶. Importantly, AIBP/apoA-I and anti-VEGF combination potently inhibited arteriolar CNV and converted it to smaller size capillary CNV (FIG. 22B). Macrophage depletion in old mice converted arteriolar CNV in old mice to capillary CNV⁶ and restores CNV sensitivity to anti-VEGF treatment (FIG. 15F)⁵, suggesting that cholesterol-laden old macrophages play a key role in forming arteriolar CNV and conferring anti-VEGF resistance in old mice. Collectively, these studies provide a strong rationale to use aged mice to model anti-VEGF resistance in CNV and also to target macrophages as a therapeutic approach.

AIBP Alone is Insufficient to Treat CNV

Although AIBP has been associated with treatment of macular degeneration or wet macular degeneration (WO 2014/193822), AIBP alone is not effective in treating at least wet AMD, as demonstrated by the inability of AIBP to suppress laser-induced CNV in mice (FIG. 15B) and that both AIBP and apoA-I are required to efficiently suppress CNV. This result is consistent with in vitro data showing that AIBP alone is insufficient to disrupt vascular tube formation by HRMECs and both AIBP and High Density Lipoprotein (HDL3) are required to efficiently suppress HTMEC vascular tube (FIG. 10C). In specific embodiments, one utilizes AIBP+apoA-I+anti-VEGF for overcoming anti-VEGF resistance.

### REFERENCES FOR THE PRESENT EXAMPLE

1. Kaiser, P. K. et al. Ranibizumab for predominantly classic neovascular age-related macular degeneration: subgroup analysis of first-year ANCHOR results. Am. J. Ophthalmol. 144, 850-857 (2007).
2. Finger, R. P., Wickremasinghe, S. S., Baird, P. N. & Guymer, R. H. Predictors of anti-VEGF treatment response in neovascular age-related macular degeneration. Surv. Ophthalmol. 59, 1-18 (2014).
3. Boyer, D. S. et al. Subgroup analysis of the MARINA study of ranibizumab in neovascular age-related macular degeneration. Ophthalmology 114, 246-252 (2007).
4. Rosenfeld, P. J. et al. Characteristics of Patients Losing Vision after 2 Years of Monthly Dosing in the Phase III Ranibizumab Clinical Trials. Ophthalmology 118, 523-530 (2011).
5. Zhu, L. et al. Combination of apolipoprotein-A-I/apolipoprotein-A-I binding protein and anti-VEGF treatment overcomes anti-VEGF resistance in choroidal neovascularization in mice. Commun. Biol. 3, 386 (2020).
6. Mettu, P. S., Allingham, M. J. & Cousins, S. W. Incomplete response to Anti-VEGF therapy in neovascular AMD: Exploring disease mechanisms and therapeutic opportunities. Prog. Retin. Eye Res. 100906 (2020) doi:10.1016/j.preteyeres.2020.100906.

### *************************

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

1. Wong, W. L. et al. Global prevalence of age-related macular degeneration and disease burden projection for 2020 and 2040: a systematic review and meta-analysis. Lancet Glob. Health 2, e106-e116 (2014).
2. Bressler, N. M., Bressler, S. B. & Fine, S. L. Age-related macular degeneration. Surv. Ophthalmol. 32, 375-413 (1988).
3. Yang, S., Zhao, J. & Sun, X. Resistance to anti-VEGF therapy in neovascular age-related macular degeneration: a comprehensive review. Drug Des. Devel. Ther. 10, 1857-1867 (2016).
4. Ehlken, C. et al. Switch of anti-VEGF agents is an option for nonresponders in the treatment of AMD. Eye Lond. Engl. 28, 538-545 (2014).
5. Heier, J. S. et al. Intravitreal aflibercept (VEGF trap-eye) in wet age-related macular degeneration. Ophthalmology 119, 2537-2548 (2012).
6. Rofagha, S. et al. Seven-year outcomes in ranibizumab-treated patients in ANCHOR, MARINA, and HORIZON: a multicenter cohort study (SEVEN-UP). Ophthalmology 120, 2292-2299 (2013).
7. Forooghian, F., Cukras, C., Meyerle, C. B., Chew, E. Y. & Wong, W. T. Tachyphylaxis after intravitreal bevacizumab for exudative age-related macular degeneration. Retina Phila. Pa. 29, 723-731 (2009).
8. Sene, A. et al. Impaired cholesterol efflux in senescent macrophages promotes age-related macular degeneration. Cell Metab. 17, 549-561 (2013).
9. Espinosa-Heidmann, D. G. et al. Macrophage depletion diminishes lesion size and severity in experimental choroidal neovascularization. Invest. Ophthalmol. Vis. Sci. 44, 3586-3592 (2003).
10. Sakurai, E., Anand, A., Ambati, B. K., van Rooijen, N. & Ambati, J. Macrophage depletion inhibits experimental choroidal neovascularization. Invest Ophthalmol Vis Sci 44, 3578-85
11. Ishida, S. et al. VEGF164-mediated inflammation is required for pathological, but not physiological, ischemia-induced retinal neovascularization. J Exp Med 198, 483-9 (2003).
12. Nagineni, C. N., Kommineni, V. K., William, A., Detrick, B. & Hooks, J. J. Regulation of VEGF expression in human retinal cells by cytokines: implications for the role of inflammation in age-related macular degeneration. J. Cell. Physiol. 227, 116-126 (2012).
13. Fang, L. et al. Control of angiogenesis by AIBP-mediated cholesterol efflux. Nature 498, 118-122 (2013).
14. Schneider, D. A. et al. AIBP protects against metabolic abnormalities and atherosclerosis. J. Lipid Res. (2018) doi:10.1194/jlr.M083618.
15. Zhang, M. et al. AIBP reduces atherosclerosis by promoting reverse cholesterol transport and ameliorating inflammation in apoE−/−mice. Atherosclerosis (2018) doi:10.1016/j.atherosclerosis.2018.03.010.
16. Makin, R. D. et al. RF/6A Chorioretinal Cells Do Not Display Key Endothelial Phenotypes. Invest. Ophthalmol. Vis. Sci. 59, 5795-5802 (2018).
17. Maekawa, M. Domain 4 (D4) of Perfringolysin O to Visualize Cholesterol in Cellular Membranes—The Update. Sensors 17, (2017).
18. Fessler, M. B. & Parks, J. S. Intracellular lipid flux and membrane microdomains as organizing principles in inflammatory cell signaling. J. Immunol. Baltim. Md. 1950 187, 1529-1535 (2011).
19. Ohtani, Y., Irie, T., Uekama, K., Fukunaga, K. & Pitha, J. Differential effects of alpha-, betaand gamma-cyclodextrins on human erythrocytes. Eur. J. Biochem. 186, 17-22 (1989).
20. Shao, Z. et al. Choroid sprouting assay: an ex vivo model of microvascular angiogenesis. PloS One 8, e69552 (2013).
21. Mills, G. L. & Taylaur, C. E. The distribution and composition of serum lipoproteins in eighteen animals. Comp. Biochem. Physiol. Part B Comp. Biochem. 40, 489-501 (1971).
22. Woller, S. A. et al. Inhibition of Neuroinflammation by AIBP: Spinal Effects upon Facilitated Pain States. Cell Rep. 23, 2667-2677 (2018).
23. Mao, R. et al. AIBP Limits Angiogenesis Through γ-Secretase-Mediated Upregulation of Notch Signaling. Circ. Res. (2017) doi:10.1161/CIRCRESAHA.116.309754.
24. Gong, Y. et al. Optimization of an Image-Guided Laser-Induced Choroidal Neovascularization Model in Mice. PloS One 10, e0132643 (2015).
25. Wang, F. et al. RNAscope: a novel in situ RNA analysis platform for formalin-fixed, paraffin-embedded tissues. J. Mol. Diagn. JMD 14, 22-29 (2012).
26. Campa, C. et al Inflammatory mediators and angiogenic factors in choroidal neovascularization: pathogenetic interactions and therapeutic implications. Mediators Inflamm. 2010, (2010).
27. Apte, R. S., Richter, J., Herndon, J. & Ferguson, T. A. Macrophages inhibit neovascularization in a murine model of age-related macular degeneration. PLoS Med. 3, e310 (2006).
28. Cherepanoff, S., McMenamin, P., Gillies, M. C., Kettle, E. & Sarks, S. H. Bruch's membrane and choroidal macrophages in early and advanced age-related macular degeneration. Br. J. Ophthalmol. 94, 918-925 (2010).
29. Grossniklaus, H. E. et al. Macrophage and retinal pigment epithelium expression of angiogenic cytokines in choroidal neovascularization. Mol. Vis. 8, 119-126 (2002).
30. Hagbi-Levi, S. et al. Proangiogenic characteristics of activated macrophages from patients with age-related macular degeneration. Neurobiol. Aging 51, 71-82 (2017).
31. Killingsworth, M. C., Sarks, J. P. & Sarks, S. H. Macrophages related to Bruch's membrane in age-related macular degeneration. Eye Lond. Engl. 4 (Pt 4), 613-621 (1990).
32. Lopez, P. F., Lambert, H. M., Grossniklaus, H. E. & Sternberg, P. Well-defined subfoveal choroidal neovascular membranes in age-related macular degeneration. Ophthalmology 100, 415-422 (1993).
33. Sarks, J. P., Sarks, S. H. & Killingsworth, M. C. Morphology of early choroidal neovascularisation in age-related macular degeneration: correlation with activity. Eye Lond. Engl. 11 (Pt 4), 515-522 (1997).
34. Kamei, M. et al. Scavenger receptors for oxidized lipoprotein in age-related macular degeneration. Invest. Ophthalmol. Vis. Sci. 48, 1801-1807 (2007).
35. Tatar, O. et al. Effect of bevacizumab on inflammation and proliferation in human choroidal neovascularization. Arch. Ophthalmol. Chic. Ill. 1960 126, 782-790 (2008).
36. Grossniklaus, H. E. et al. Correlation of histologic 2-dimensional reconstruction and confocal scanning laser microscopic imaging of choroidal neovascularization in eyes with age-related maculopathy. Arch. Ophthalmol. Chic. Ill. 1960 118, 625-629 (2000).
37. Nishimura, T., Goodnight, R., Prendergast, R. A. & Ryan, S. J. Activated macrophages in experimental subretinal neovascularization. Ophthalmol. J. Int. Ophtalmol. Int. J. Ophthalmol. Z. Augenheilkd. 200, 39-44 (1990).
38. Oh, H. et al. The potential angiogenic role of macrophages in the formation of choroidal neovascular membranes. Invest. Ophthalmol. Vis. Sci. 40, 1891-1898 (1999).
39. Pollack, A., Korte, G. E., Heriot, W. J. & Henkind, P. Ultrastructure of Bruch's membrane after krypton laser photocoagulation. II. Repair of Bruch's membrane and the role of macrophages. Arch. Ophthalmol. Chic. Ill. 1960 104, 1377-1382 (1986).
40. Krzystolik, M. G. et al. Prevention of experimental choroidal neovascularization with intravitreal anti-vascular endothelial growth factor antibody fragment. Arch. Ophthalmol. Chic. Ill. 1960 120, 338-346 (2002).
41. Zhang, X., Goncalves, R. & Mosser, D. M. The Isolation and Characterization of Murine Macrophages. Curr. Protoc. Immunol. Ed. John E Coligan A1 CHAPTER, Unit-14.1 (2008).
42. Espinosa-Heidmann, D. G. et al. Age as an independent risk factor for severity of experimental choroidal neovascularization. Invest. Ophthalmol. Vis. Sci. 43, 1567-1573 (2002).
43. Espinosa-Heidmann, D. G. et al. Gender and estrogen supplementation increases severity of experimental choroidal neovascularization. Exp. Eye Res. 80, 413-423 (2005).
44. Kumar, S. et al. Proteolytic Degradation and Inflammation Play Critical Roles in Polypoidal Choroidal Vasculopathy. Am. J. Pathol. 187, 2841-2857 (2017).

What is claimed is:

1. A method of treating or preventing neovascularization in an individual, comprising the step of delivering to the individual a therapeutically effective amount of a composition comprising apoA-I binding protein (AIBP) or a functionally active fragment or derivative thereof.

2. The method of claim 1, wherein the neovascularization is associated with age-related macular degeneration.

3. The method of claim 1, wherein the neovascularization is associated with cancer.

4. The method of any one of the preceding claims, wherein the neovascularization is associated with resistance to anti-VEGF agents.

5. The method of any one of the preceding claims, wherein the neovascularization is associated with aberrant new blood vessel formation.

6. The method of any one of the preceding claims, wherein the fragment comprises the N-terminus, the C-terminus, both the N-terminus and C-terminus, or neither of the N-terminus or C-terminus.

7. The method of any one of the preceding claims, wherein the fragment or derivative is at least 70, 75, 80, 85, 90, 91, 92, 93, 94, 95, 96, 97, 98, or 99% identical to SEQ ID NO:1.

8. The method of any one of the preceding claims, wherein the derivative comprises 1, 2, 3, 4, 5, or more variations compared to SEQ ID NO:1.

9. The method of any one of the preceding claims, wherein treatment with the AIBP composition improves removal of cholesterol from endothelial cells and/or macrophages, reduces inflammation, and restores macrophages' ability to inhibit angiogenesis, thereby treating or preventing neovascularization.

10. The method of any one of the preceding claims, wherein the AIBP composition is delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage.

11. The method of any one of the preceding claims, wherein the AIBP composition is delivered to the individual multiple times.

12. The method of claim 11, wherein the AIBP composition is delivered to the individual once a day, more than once a day, more than once a week, more than once a month, or more than once a year.

13. The method of any one of the preceding claims, wherein the AIBP composition is provided to the individual by constant infusion.

14. The method of any one of the preceding claims, wherein provision of the AIBP composition reduces resistance to anti-VEGF agents.

15. The method of any one of the preceding claims, wherein the individual is provided one or more additional therapies for treating or preventing neovascularization.

16. The method of claim 14, wherein the second therapy comprises an anti-VEGF agent.

17. The method of claim 15, wherein the anti-VEGF agent comprises one or more antibodies selected from the group consisting of brolucizumab, pegaptanib, bevacizumab, conbercept, ranibizumab, and afilbercept.

18. The method of claim 15, wherein the anti-VEGF agent comprises one or more small molecules selected from the group consisting of lapatinib, sunitinib, sorafenib, axitinib, pazopanib, and AZ2171 (cediranib).

19. The method of claim 15, wherein the anti-VEGF agent comprises AAV2-sFLT-1 or AAV2-sFLT01.

20. The method of any of claims 14-19, wherein the anti-VEGF agent is provided before, during, or after provision of the AIBP composition.

21. The method of any one of the preceding claims, further comprising the step of administering to the individual an effective amount of apoA-1.

22. The method of claim 21, wherein the apoA-1 is delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage.

23. The method of any one of the preceding claims, wherein the apoA-1 is delivered to the individual once or multiple times.

24. The method of any of claims 21-23, wherein the apoA-1 is provided before, during, or after provision of the AIBP composition.

25. The method of any of claims 21-24, wherein the apoA-1 is provided before, during, or after provision of the anti-VEGF agent.

26. A method of treating or preventing pathological neovascularization in an individual, comprising the step of delivering to the individual a therapeutically effective amount of an anti-apoA-I binding protein (AIBP) agent.

27. The method of claim 26, wherein the neovascularization is associated with ischemic retinopathy.

28. The method of claim 26 or 27, wherein the ischemic retinopathy is retinopathy of prematurity (ROP), diabetic retinopathy (DR), or central retinal vein occlusion.

29. The method of any one of claims 26-28, wherein the neovascularization is associated with aberrant new blood vessel formation in the vitreous humor.

30. The method of any one of claims 26-29, wherein treatment with the anti-AIBP agent inhibits AIBP.

31. The method of claim 30, wherein inhibition of AIBP promotes VEGFR2 signaling and inhibits Notch1 signaling.

32. The method of claim 30 or 31, wherein treatment with the anti-AIBP agent inhibits pathological neovascularization.

33. The method of any one of claims 26-32, wherein treatment with the anti-AIBP agent promotes regenerative revascularization.

34. The method of any one of claims 26-33, wherein the anti-AIBP agent is delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage.

35. The method of any one of claims 26-34, wherein the anti-AIBP agent is delivered to the individual multiple times.

36. The method of claim 35, wherein the anti-AIBP agent is delivered to the individual once a day, more than once a day, more than once a week, more than once a month, or more than once a year.

37. The method of any one of claims 26-36, wherein the anti-AIBP agent is provided to the individual by constant infusion.

38. The method of any one of claims 26-37, wherein the anti-AIBP agent comprises anti-AIBP antibodies, antisense nucleotides, blocking peptides, and/or small molecule antagonists of AIBP.

39. A method of overcoming resistance to an anti-VEGF therapy in an individual, comprising the step of providing to the individual an effective amount of a composition comprising AIBP or a functionally active fragment or derivative thereof.

40. The method of claim 39, wherein the fragment comprises the N-terminus, the C-terminus, both the N-terminus and C-terminus, or neither of the N-terminus or C-terminus.

41. The method of claim 39 or 40, wherein the fragment or derivative is at least 70, 75, 80, 85, 90, 91, 92, 93, 94, 95, 96, 97, 98, or 99% identical to SEQ ID NO:1.

42. The method of any of claims 39-41, wherein the derivative comprises 1, 2, 3, 4, 5, or more variations compared to SEQ ID NO:1.

43. The method of any of claims 39-42, wherein the AIBP composition is delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage.

44. The method of any of claims 39-43, wherein the AIBP composition is delivered to the individual multiple times.

45. The method of claim 44, wherein the AIBP composition is delivered to the individual once a day, more than once a day, more than once a week, more than once a month, or more than once a year.

46. The method of any of claims 39-45, wherein the AIBP composition is provided to the individual by constant infusion.

47. The method of any of claims 39-46, further comprising providing to the individual an effective amount of one or more anti-VEGF agents.

48. The method of claim 47, wherein the anti-VEGF agent is provided before, during, or after provision of the AIBP composition.

49. The method of any of claims 39-48, wherein the AIBP composition further comprises one or more anti-VEGF agents.

50. The method of any of claims 39-49, wherein the individual is determined to be at risk of having resistance to one or more anti-VEGF agents.

51. The method of claim 50, wherein an individual at risk of having resistance to one or more anti-VEGF agents exhibits an initial lesion with subfoveal fibrosis and/or atrophy in retina pigment epithelium and photoreceptors, lesion in large size, type 1 choroidal neovascularization, serous pigment epithelium detachment (PED), hemorrhagic PED, fibrovascular PED, polypoidal choroidal vasculopathy, foveal scarring and vitreomacular traction, outer retinal tubulation, cystoid degeneration in outer retina, and/or a genetic disposition to resistance.

52. The method of claim 45, wherein the AIBP or a functionally active fragment or derivative thereof and, optionally, one or more anti-VEGF agents are provided to an individual when the individual has one or more risk factors for developing resistance to one or more anti-VEGF agents.

53. The method of claim 52, wherein the AIBP or a functionally active fragment or derivative thereof and, optionally, one or more anti-VEGF agents are provided to an individual regardless of whether resistance to one or more anti-VEGF agents has been demonstrated.

54. The method of any of claims 39-53, wherein the individual is determined to have resistance to one or more anti-VEGF agents.

55. The method of any of claims 50-54, wherein the anti-VEGF agent comprises one or more antibodies selected from the group consisting of brolucizumab, pegaptanib, bevacizumab, conbercept, ranibizumab, and afilbercept.

56. The method of any of claims 50-54, wherein the anti-VEGF agent comprises one or more small molecules selected from the group consisting of lapatinib, sunitinib, sorafenib, axitinib, pazopanib, and AZ2171 (cediranib).

57. The method of any of claims 50-54, wherein the anti-VEGF agent comprises AAV2-sFLT-1 or AAV2-sFLT01.

58. The method of any of claims 50-57, wherein provision of the AIBP or a functionally active fragment or derivative thereof and one or more anti-VEGF agents has an additive or synergistic therapeutic effect in the individual.

59. The method of any one of claims 39-58, further comprising the step of administering to the individual an effective amount of apoA-1.
